(19) United States
(12) Patent Application Publication
(10) Pub. No.: US 2026/0301969 A1
(43) Pub. Date: Oct. 1, 2026

(54) ELECTRONIC DEVICE AND DATA MANAGEMENT METHOD OF ELECTRONIC DEVICE

(71) Applicant: SAMSUNG ELECTRONICS CO., LTD., Suwon-si (KR)

(72) Inventors: Sunghwan CHO, Suwon-si (KR); Jeongmin PARK, Suwon-si (KR); Jaepil KIM, Suwon-si (KR); Byeongju PARK, Suwon-si (KR); Jaeyung YEO, Suwon-si (KR); Myeongjin LEE, Suwon-si (KR)

(21) Appl. No.: 19/679,251

(22) Filed: May 15, 2026

Related U.S. Application Data
(63) Continuation of application No. PCT/KR2024/015817, filed on Oct. 17, 2024.

(30) Foreign Application Priority Data
Nov. 17, 2023 (KR) 10-2023-0159738
Dec. 27, 2023 (KR) 10-2023-0193627

(51) Int. Cl. G16H 50/30; G06F 3/01

(52) U.S. Cl. CPC G16H 50/30; G06F 3/015

(57) ABSTRACT

An electronic device according to an embodiment may include communication circuitry, a display, at least one sensor, a memory including a generative artificial intelligence model, and a processor. The memory may store instructions that, when executed by the processor, cause the electronic device to identify biometric data periodically measured using the at least one sensor, determine whether the biometric data is missing, identify information related to a period during which the biometric data is missing, identify a measurement history of previously measured biometric data, and generate, using the generative artificial intelligence model, missing biometric data based at least in part on the measurement history.

### CROSS-REFERENCE TO RELATED APPLICATION(S)

This application is a continuation application, claiming priority under § 365 (c), of International Application No. PCT/KR2024/015817, filed on Oct. 17, 2024, which is based on and claims the benefit of Korean patent application number 10-2023-0193627 filed on Dec. 27, 2023, in the Korean Intellectual Property Office and of Korean patent application number 10-2023-0159738, filed on Nov. 17, 2023, in the Korean Intellectual Property Office, the disclosures of which are incorporated by reference herein in their entireties.

### TECHNICAL FIELD

Embodiments disclosed herein relate to technology for detecting whether data obtained using a sensor is missing and for managing the data.

### BACKGROUND ART

Recent portable electronic devices, such as mobile terminals and wearable devices, may provide various functions in addition to communication with external electronic devices. For example, as smartphones, wearable devices, and other portable electronic devices have become widespread, methods for measuring biometric information using such electronic devices have been studied. A portable electronic device may include a sensor configured to measure biometric information. A plurality of portable electronic devices, such as a mobile terminal and a wearable device, may share measured biometric information with one another. As biometric information is measured using the portable electronic device, a healthcare service may be provided to a user. For example, the portable electronic device may periodically or aperiodically measure biometric information of the user, identify a health state of the user, and provide the user with information associated with the health state.

The information described above is provided as background to aid understanding of the disclosure, and no admission is made as to whether any of the above-described information constitutes prior art to the present disclosure.

### SUMMARY

According to an embodiment, an electronic device may include communication circuitry, a display, at least one sensor, a memory including a generative artificial intelligence model, and a processor. The memory may store instructions that, when executed by the processor, cause the electronic device to identify biometric data periodically measured using the at least one sensor, determine whether the biometric data is missing, identify information related to a period during which the biometric data is missing, identify a measurement history of previously measured biometric data, and generate, using the generative artificial intelligence model, missing biometric data based at least in part on the measurement history.

According to an embodiment, a method for managing data of an electronic device may include identifying biometric data periodically measured using at least one sensor, determining whether the biometric data is missing, identifying information related to a period during which the biometric data is missing, identifying a measurement history of previously measured biometric data, and generating, using a generative artificial intelligence model, missing biometric data based at least in part on the measurement history.

According to an embodiment, a storage medium may store instructions or a program that, when executed by a processor of an electronic device, cause the electronic device to identify biometric data periodically measured using at least one sensor, determine whether the biometric data is missing, identify information related to a period during which the biometric data is missing, identify a measurement history of previously measured biometric data, and generate, using a generative artificial intelligence model, missing biometric data based at least in part on the measurement history.

### DESCRIPTION OF DRAWINGS

FIG. 1 is a block diagram of an electronic device according to an embodiment;

FIG. 2 is a block diagram of a system according to an embodiment;

FIG. 3 is a block diagram of an electronic device according to an embodiment;

FIG. 4 is a drawing for describing an operation of generating missing biometric data in an electronic device according to an embodiment;

FIG. 5 is a drawing for describing an operation of detecting that data is missing in an electronic device according to an embodiment;

FIGS. 6A and 6B are drawings for describing an operation of generating and providing missing data in an electronic device according to an embodiment;

FIG. 7 is a drawing for describing an operation of generating and providing missing data in an electronic device according to an embodiment;

FIG. 8 illustrates an example of a user interface provided to manage data by an electronic device according to an embodiment;

FIG. 9 is a drawing for describing an operation of generating missing sensor data in an electronic device according to an embodiment;

FIG. 10 is a flowchart of a method for managing data of an electronic device according to an embodiment;

FIG. 11 is a flowchart of a method for managing data of an electronic device according to an embodiment;

FIG. 12 is a flowchart of a method for managing data of an electronic device according to an embodiment;

FIG. 13 is a flowchart of a method for managing data of an electronic device according to an embodiment;

FIG. 14 is a flowchart of a method for managing data of an electronic device according to an embodiment; and

FIG. 15 is a block diagram illustrating an electronic device in a network environment according to various embodiments.

In the drawings, the same or similar components may be denoted by the same or similar reference numerals.

### DETAILED DESCRIPTION

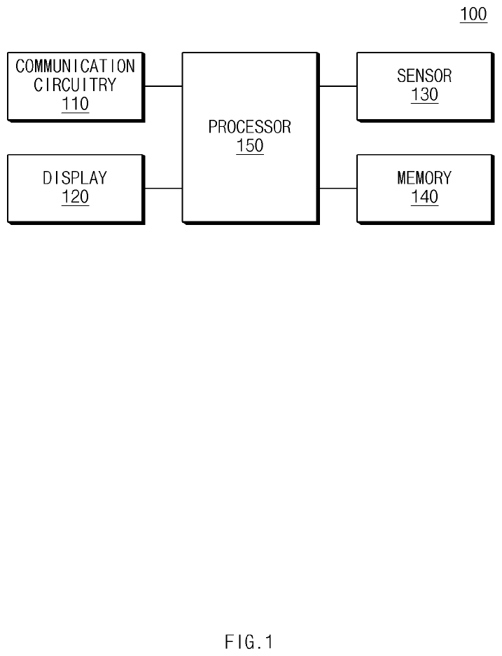

FIG. 1 is a block diagram of an electronic device according to an embodiment.

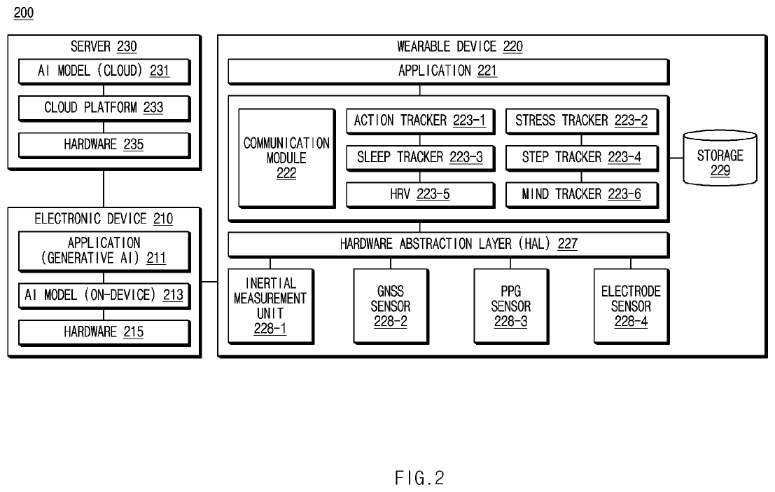

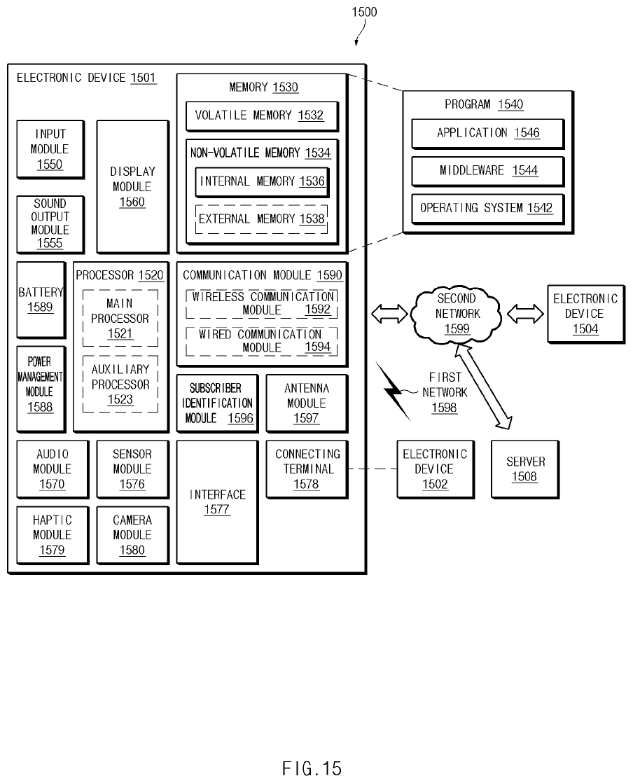

According to an embodiment, an electronic device 100, for example, an electronic device 210 of FIG. 2 or an electronic device 1501 of FIG. 15, may include communication circuitry 110, for example, a communication module 1590 of FIG. 15, a display 120, for example, a display module 1560 of FIG. 15, at least one sensor 130, for example, sensors 228-1, 228-2, 228-3, and 228-4 of FIG. 2 or a sensor module 1576 of FIG. 15, a memory 140, for example, a memory 1530 of FIG. 15, and a processor 150, for example, a processor 1520 of FIG. 15.

According to an embodiment, the communication circuitry 110 may transmit and receive information and/or data to and from an external electronic device, for example, an electronic device 1502 or 1504 of FIG. 15, and/or an external server, for example, a server 1508 of FIG. 15. For example, the external electronic device may include a component substantially the same as the electronic device 100 and may include a component different from the electronic device 100. For example, the communication circuitry 110 may receive biometric data of a user and/or sensor data obtained by the external electronic device. For example, the communication circuitry 110 may transmit the biometric data and/or the sensor data to the external server and may receive missing biometric data and/or missing sensor data, generated by the external server, for example, by a generative artificial intelligence model of the external server, from the external server.

According to an embodiment, the display 120 may visually output information. For example, the display 120 may output the biometric data and/or the sensor data. For example, the display 120 may output an execution screen of an application and/or a user interface including the biometric data and/or the sensor data. For example, the display 120 may output a user interface including generated missing data. According to an embodiment, the display 120 may output a user interface for confirming whether to store the generated missing data.

According to an embodiment, the at least one sensor 130 may measure biometric information of the user. For example, the at least one sensor 130 may be included in an exterior portion of the electronic device 100 or in an external electronic device.

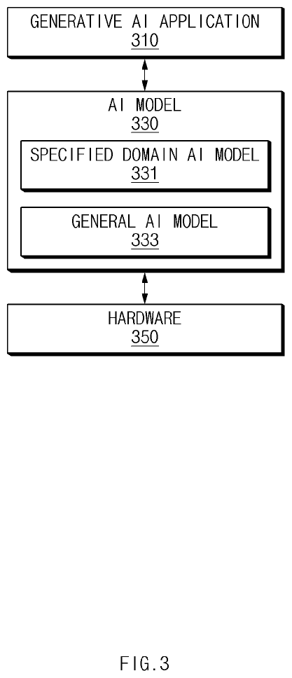

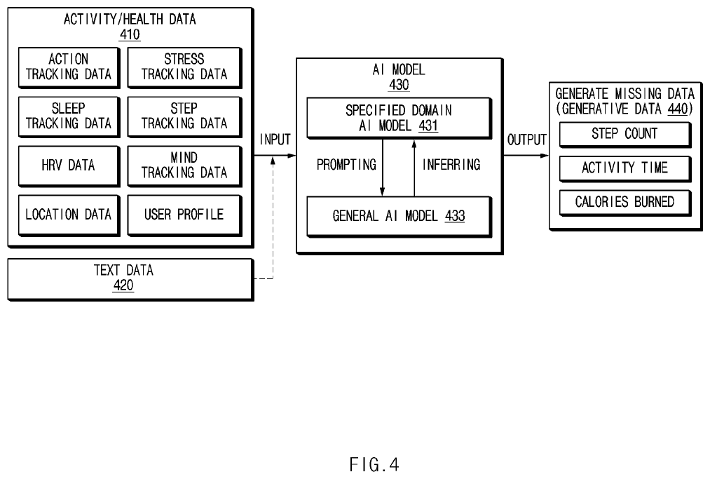

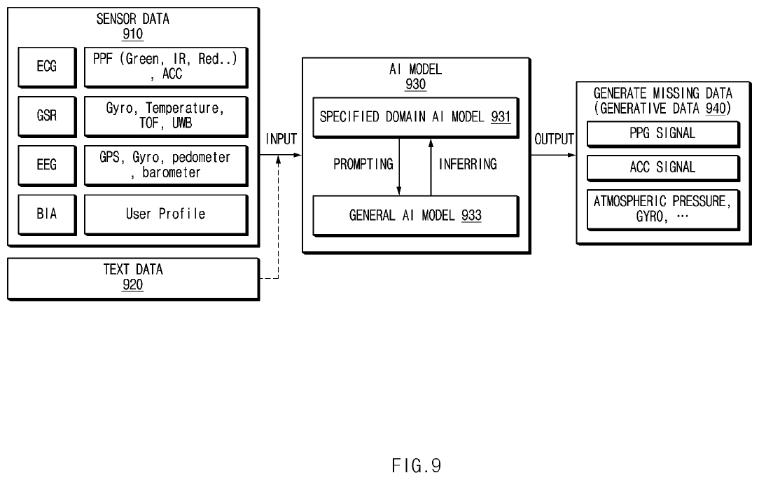

According to an embodiment, the memory 140 may store instructions that, when executed by the processor 150, control an operation of the electronic device 100. The memory 140 may include a generative artificial intelligence model, for example, an AI model 213 of FIG. 2, an AI model 330 of FIG. 3, an AI model 430 of FIG. 4, or an AI model 930 of FIG. 9. The memory 140 may store sensor data and/or biometric data of the user measured using the at least one sensor 130 of the electronic device 100 and/or an external electronic device. The memory 140 may store a user profile, for example, a health state, body information, a name, a gender, an age, a preference of the user, activity history, and/or device usage history. The memory 140 may at least temporarily store text data obtained from a user input.

According to an embodiment, the processor 150 may identify biometric data periodically or aperiodically measured using the at least one sensor 130. For example, the electronic device 100 may periodically or aperiodically measure biometric data. The electronic device 100 may store biometric data measured using the at least one sensor 130 and May identify the stored biometric data. For example, a measurement period of the biometric data may include, but is not limited to, one day, one month, or one year, and may be specified as any suitable period. According to an embodiment, the processor 150 may receive biometric data measured using a sensor of an external electronic device, for example, a wearable device, from the external electronic device.

According to an embodiment, the processor 150 may determine whether the biometric data is missing. For example, when the period is one day, the processor 150 may identify a day on which the biometric data is missing, hereinafter referred to as a “missing day.” According to an embodiment, the processor 150 may determine whether the biometric data is missing based on a result of comparing, for each specified cycle, a time at which the biometric data is measured, an operation state of the electronic device 100, for example, a time at which the electronic device 100 is powered on, a time at which the electronic device 100 is powered off, or a time at which the at least one sensor 130 is activated or deactivated, or a time during which the external electronic device, for example, the wearable device, is worn, with a specified threshold, for example, a threshold time.

According to an embodiment, the processor 150 may identify information related to a period during which the biometric data is missing. For example, the processor 150 may identify information associated with a missing day of the biometric data, for example, a date, weather, a day of the week, and/or a year.

According to an embodiment, the processor 150 may identify a measurement history of previously measured biometric data. For example, the processor 150 may identify a measurement history of biometric data measured before the missing day and/or a measurement history of biometric data measured after the missing day. For example, the processor 150 may identify a measurement history of biometric data measured during another period corresponding to the missing period.

According to an embodiment, the processor 150 may determine whether additional data is required to generate the missing biometric data. For example, when the additional data is required to generate the missing biometric data, the processor 150 may receive text data associated with behavior of the user during the period from the user. According to an embodiment, the processor 150 may obtain the text data from the user through an input via a user interface, an input via a physical key, and/or a voice input via a microphone.

According to an embodiment, the processor 150 may generate the missing biometric data, using the generative artificial intelligence model, based at least in part on the measurement history of the biometric data and/or the text data.

According to an embodiment, the processor 150 may obtain sensor data measured using a plurality of sensors 130 during a specified period, which may be referred to as a “session” in the disclosure. For example, the specified period may be set as a different period based on a user input, a state of the electronic device 100, for example, a log, an application execution state, and/or a state of the user, for example, an exercise state, a sleep state, or an activity state, as determined by the electronic device 100.

According to an embodiment, the processor 150 may determine whether sensor data corresponding to at least one sensor 130 is missing. For example, the processor 150 may determine whether data of the at least one sensor 130 among the plurality of sensors 130 used to generate information, for example, exercise information or health information, associated with the user during the specified period is missing. According to an embodiment, the processor 150 may identify information associated with a session in which the sensor data is missing, hereinafter referred to as a “missing session,” for example, a start time, an end time, a duration of the missing session, and/or a state of the user associated with the session, for example, a sleep state, an exercise state, an activity state, and/or whether the user is indoors or outdoors.

According to an embodiment, the processor 150 may identify measured sensor data excluding the missing sensor data during the specified period, for example, the missing period. For example, the processor 150 may identify sensor data measured by other sensors 130 except for the sensor 130 corresponding to the missing sensor data among the plurality of sensors 130.

According to an embodiment, the processor 150 may identify a measurement history of previously measured sensor data. For example, the processor 150 may identify a measurement history of sensor data measured during a previous period corresponding to the specified period, that is, the missing session, for example, a session different from the missing session. According to an embodiment, the processor 150 may determine whether additional data is required to generate the missing sensor data. For example, when the additional data is required to generate the missing sensor data, the processor 150 may receive text data associated with behavior of the user during the specified period from the user. The processor 150 may obtain the text data from the user through an input via the user interface, an input via a physical key, and/or a voice input via the microphone.

According to an embodiment, the processor 150 may generate the missing sensor data based at least in part on the measured sensor data, using the generative artificial intelligence model. According to an embodiment, the processor 150 may generate missing biometric data based on sensor data obtained through another sensor 130 other than the sensor 130 corresponding to the missing sensor data in the same session, sensor data obtained in a different session, and/or text data obtained from the user.

According to an embodiment, the processor 150 may obtain sensor data measured using the at least one sensor 130. For example, the processor 150 may obtain the sensor data using the at least one sensor 130 included in the electronic device 100 or may receive the sensor data from an external electronic device.

According to an embodiment, the processor 150 may set a parameter corresponding to a specified condition. For example, the processor 150 may set a parameter corresponding to a virtual health state.

According to an embodiment, the processor 150 may generate sensor data under the specified condition based on the parameter and the sensor data, using the generative artificial intelligence model. According to an embodiment, the processor 150 may apply the set parameter to measured sensor data to generate sensor data under a specified virtual condition, using the generative artificial intelligence model. For example, the processor 150 may generate sensor data corresponding to a virtual exercise state, for example, anaerobic exercise, aerobic exercise, strength exercise, exercise with a relatively large amount of movement, or exercise with a relatively small amount of movement, using the generative artificial intelligence model.

FIG. 1 illustrates a case in which the electronic device 100 performs both an operation of measuring data and an operation of generating missing data. However, according to various embodiments, the electronic device 100 may perform the operation of measuring data and/or the operation of generating missing data in conjunction with an external electronic device and/or an external server. For example, the electronic device 100 may receive biometric data and/or sensor data obtained using a sensor 130 included in an external electronic device, for example, a wearable device, from the external electronic device. The electronic device 100 may generate missing data at least in part in conjunction with the external server, for example, a generative artificial intelligence model of the external server.

According to an embodiment, the configuration of the electronic device 100 is not limited to that illustrated in FIG. 1, and some configurations may be omitted or at least one configuration may be added, for example, the electronic device 210 and the wearable device 220 of FIG. 2 and/or at least one of the components of the electronic device 1501 of FIG. 15.

According to an embodiment, the electronic device 100 may identify that biometric data and/or sensor data is missing during a specified cycle or a specified period and May generate the missing biometric data and/or sensor data using the generative artificial intelligence model. According to an embodiment, the electronic device 100 may generate sensor data corresponding to a virtual condition in various ways such that sensor data usable to develop, analyze, and/or evaluate an algorithm may be generated even in a limited environment.

FIG. 2 is a block diagram of a system 200 according to an embodiment.

According to an embodiment, the system 200 may include an electronic device 210, for example, the electronic device 100 of FIG. 1 or the electronic device 1501 of FIG. 15, a wearable device 220, for example, the electronic device 1501, 1502, or 1504 of FIG. 15, and a server 230, for example, the server 1508 of FIG. 15.

According to an embodiment, the electronic device 210 may include an application 211, for example, an application 1546 of FIG. 15, an AI model 213, for example, the AI model 330 of FIG. 3, the AI model 430 of FIG. 4, or the AI model 930 of FIG. 9, and hardware 215, for example, hardware 350 of FIG. 3.

According to an embodiment, the application 211 may include a generative artificial intelligence-based application, for example, a health application, configured to provide analysis information associated with a user based on artificial intelligence. For example, the application 211 may provide the AI model 213 with at least one item of sensor data and/or biometric data associated with the user. The sensor data and/or the biometric data may include data obtained through a sensor of the electronic device 210 and/or an external electronic device, for example, the wearable device 220. For example, the application 211 may provide analysis information associated with the user through a user interface based on data provided from the AI model 213. For example, the application 211 may provide the user with guide information, such as exercise, meditation, or counseling information, based on biometric information.

According to an embodiment, the AI model 213 may include a generative artificial intelligence model. For example, the AI model 213 may include a model personalized for a particular domain and a model used when processing data requiring security, such as biometric data of the user. For example, the AI model 213 may include an artificial intelligence model for collecting, training, analyzing, and/or predicting data regardless of a specific purpose and/or domain. For example, the AI model 213 may include, but is not limited to, a large language model (LLM), a generative adversarial network, and/or a variational autoencoder. According to an embodiment, the AI model 213 may determine whether biometric data and/or sensor data is missing and may generate missing biometric data and/or missing sensor data based on a measurement history of previously obtained biometric data and/or sensor data, a user input, for example, information indicating behavior of the user during a specified cycle or a specified period, and/or non-missing biometric data and/or sensor data.

According to an embodiment, the hardware 215 may include at least one processing device, for example, a neural processing unit (NPU), a graphics processing unit (GPU), or a central processing unit (CPU), and at least one hardware component, for example, at least one sensor, communication circuitry, a memory, and/or a display. For example, the electronic device 210 may receive sensor data and/or biometric data from the wearable device 220 and may obtain biometric data of the user and/or sensor data using at least one sensor included in the electronic device 210. For example, the hardware 215 may include at least one of the components of the electronic device 1501 of FIG. 15.

According to an embodiment, the electronic device 210 may generate missing biometric data and/or missing sensor data in conjunction with a cloud platform 233 of the server 230. For example, the electronic device 210 may transmit data, for example, sensor data and/or biometric data, to the server 230 through communication circuitry of the server 230 and may receive a result of processing the data, for example, missing biometric data and/or missing sensor data generated by the server 230, from the server 230. For example, the electronic device 210 may deliver the data received from the server 230 to the application 211.

According to an embodiment, the wearable device 220 may include an application 221, for example, an application 1546 of FIG. 15, a communication module 222, for example, a communication module 1590 of FIG. 15, a plurality of measurement modules, a hardware abstraction layer (HAL) 227, a plurality of sensors, for example, a sensor module 1576 of FIG. 15, and storage 229, for example, a memory 1530 of FIG. 15.

According to an embodiment, the application 221 may provide information associated with the user, for example, activity information, health information, sleep information, and/or exercise information, based on data provided from the plurality of measurement modules.

According to an embodiment, the communication module 222 may transmit and receive information and/or data to and from an external electronic device, for example, the electronic device 210 and/or the server 230. For example, the communication module 222 may transmit biometric data obtained using the plurality of measurement modules and/or sensor data obtained using the plurality of sensors to the electronic device 210.

According to an embodiment, the plurality of measurement modules may generate biometric data associated with the user. The plurality of measurement modules may measure, track, monitor, and/or analyze the biometric data associated with the user. According to an embodiment, the plurality of measurement modules may include an action tracker 223-1, a stress tracker 223-2, a sleep tracker 223-3, a step tracker 223-4, a heart rate variability (HRV) module 223-5, and a mind tracker 223-6.

For example, the action tracker 223-1 may measure the number of actions of the user per specified time, for example, per minute. For example, the action tracker 223-1 may track an action of the user that does not have a specific pattern. The action tracker 223-1 may measure a degree, continuity, strength, and/or a displacement difference of the action of the user through a sensor, for example, an inertial measurement unit 228-1.

The stress tracker 223-2 may determine stress of the user based on data provided from the HRV module 223-5. For example, the stress tracker 223-2 may determine or classify a stress index of the user. For example, the stress tracker 223-2 may classify the stress of the user as acute stress or chronic stress and may determine and provide a stress index. The stress tracker 223-2 may measure electrodermal activity (EDA) using a sensor, for example, an electrode sensor 228-4, and may determine stress of the user based on skin tension. The stress tracker 223-2 may synthetically analyze HRV information, inter-beat interval (IBI) information, and various items of biometric information and may determine a stress index of the user and/or whether the user has acute stress or chronic stress.

The sleep tracker 223-3 may track a sleep state or a sleep pattern of the user. The sleep tracker 223-3 may measure a sleep posture of the user through a sensor, for example, the inertial measurement unit 228-1. For example, the sleep tracker 223-3 may analyze a sleep state of the user based on data measured by the HRV module 223-5 during sleep. For example, the sleep tracker 223-3 may extract sympathetic nerve and parasympathetic nerve components through frequency analysis of sensor data and may determine a sleep state of the user, for example, an awake state, a state just before sleep, a state just after sleep, light sleep, deep sleep, or rapid eye movement (REM) sleep.

The step tracker 223-4 may measure a step count of the user based on an impulse value detected while the user is walking, as measured using a sensor, for example, an accelerometer sensor. For example, the step tracker 223-4 may identify an impulse value or a step frequency while the user is moving and may generate subdivided analysis information, such as a walking state, for example, walking or running, of the user. For example, when identifying a step pattern of at least a specified value, the step tracker 223-4 may measure a step count of the user to increase the accuracy of measuring the step count of the user. The step tracker 223-4 may provide information associated with a speed at which the user is walking or running, a time at which the user moves, or a state of the user when walking or running, based on the measured information.

The HRV module 223-5 may measure regularity or variability of a heart rate of the user. For example, the HRV module 223-5 may measure a heart rate (HR) of the user. The HRV module 223-5 may identify information about a peak-to-peak inter-beat interval (IBI) of a series of heart rate information and may generate variance information, deviation information, time-series analysis information, and/or frequency analysis information from the IBI information.

The mind tracker 223-6 may determine a depression index of the user based on biometric data collected from the plurality of measurement modules. The mind tracker 223-6 may determine the depression index based on information input from the user. The mind tracker 223-6 may track and observe the depression index of the user and may provide the user with a corresponding activity based thereon.

According to various embodiments, the measurement modules included in the wearable device 220 are not limited to those illustrated in FIG. 2.

According to an embodiment, the HAL 227 may transmit and receive information and/or data between the application 221, the communication module 222, and/or the plurality of measurement modules and hardware, for example, the plurality of sensors. For example, the HAL 227 may control operations of the plurality of sensors in conjunction with operations of the application 221, the communication module 222, and/or the plurality of measurement modules.

According to an embodiment, the plurality of sensors may include the inertial measurement unit 228-1, the global navigation satellite system (GNSS) sensor 228-2, the photoplethysmography (PPG) sensor 228-3, and the electrode sensor 228-4.

For example, the inertial measurement unit 228-1 may include, but is not limited to, an accelerometer sensor, a gyroscope sensor, and/or a geomagnetic sensor. For example, the inertial measurement unit 228-1 may measure acceleration, direction, speed, distance change, and/or posture of the electronic device 210.

For example, the GNSS sensor 228-2 may include a global positioning system (GPS) sensor, a GLONASS sensor, a BeiDou sensor, and/or a Galileo sensor. For example, the GNSS sensor 228-2 may measure a location of the electronic device 210.

For example, the PPG sensor 228-3 may emit light of various wavelengths and May measure biometric data, for example, heart rate and/or oxygen saturation, of the user based on light reflected from a body of the user and incident on the sensor. According to an embodiment, FIG. 2 illustrates only the PPG sensor 228-3, but the wearable device 220 may include various optical sensors for biometric measurement including the PPG sensor 228-3. For example, an optical sensor may include at least one light source, for example, a light-emitting element such as an LED, for emitting light of various wavelengths. For example, green light may be used to measure heart rate and may have an advantage in that it penetrates relatively shallowly into the skin of the user and is robust to noise. For example, red light may penetrate relatively deeply into the skin of the user and may be used to measure heart rate with higher accuracy. For example, infrared light may be used to measure heart rate and oxygen saturation (SpO2). Blue light may be used to measure blood glucose of the user. For example, the optical sensor may include a light detector including at least one photodiode.

For example, the electrode sensor 228-4 may include at least one electrode. For example, the electrode sensor 228-4 may include a sensor for measuring biometric data through the electrode. For example, the electrode sensor 228-4 may measure various items of biometric data in conjunction with another sensor, for example, the PPG sensor 228-3.

According to various embodiments, the sensors included in the wearable device 220 are not limited to those illustrated in FIG. 2. For example, the wearable device 220 may further include a proximity sensor, a temperature sensor, for example, a body temperature sensor, a gas sensor, a fine dust sensor, a humidity sensor, an illumination sensor, a time-of-flight (TOF) sensor, and/or an ultra-wideband (UWB) sensor, for example, a LiDAR sensor.

The storage 229 may store data associated with an operation of the wearable device 220. For example, the storage 229 may store biometric data obtained through the plurality of measurement modules and/or sensor data obtained using the plurality of sensors.

According to an embodiment, the server 230 may include an AI model 231, a cloud platform 233, and hardware 235. According to an embodiment, the AI model 231 of the server 230 may correspond to the AI model 213 of the electronic device 210. For example, the server 230, for example, the AI model 231 of the server 230, may have fewer scale and spatial constraints than the electronic device 210 and may support a larger amount of computation using more resources to process a larger amount of data more quickly and accurately than the electronic device 210. According to an embodiment, the cloud platform 233 may transmit and receive data to and from the electronic device 210. According to an embodiment, the hardware 235 of the server 230 may include at least one processing device, for example, an NPU, a GPU, or a CPU, and at least one hardware component, for example, communication circuitry and/or a memory.

According to an embodiment, the system 200 may identify that biometric data and/or sensor data is missing during a specified cycle or a specified period and may generate and use the missing biometric data and/or sensor data using the generative artificial intelligence model.

FIG. 3 is a drawing illustrating a layer of a generative artificial intelligence model according to an embodiment. Hereinafter, content the same as or similar to the content described with reference to FIG. 2 may be omitted or briefly described.

According to an embodiment, an electronic device, for example, the electronic device 100 of FIG. 1, the electronic device 210 of FIG. 2, or the electronic device 1501 of FIG. 15, may include a generative AI application 310, for example, the application 211 of FIG. 2, an AI model 330, for example, the AI model 213 of FIG. 2, the AI model 430 of FIG. 4, or the AI model 930 of FIG. 9, and hardware 350, for example, the hardware 215 of FIG. 2.

According to an embodiment, the generative AI application 310 may include an application, for example, a health application, configured to provide analysis information associated with a user based on artificial intelligence. For example, the generative AI application 310 may provide the AI model 330 with at least one item of sensor data and/or biometric data associated with the user. For example, the generative AI application 310 may provide analysis information associated with the user through a user interface based on data provided from the AI model 330.

According to an embodiment, the AI model 330 may include a specified-domain AI model 331 and a general AI model 333. For example, the AI model 330 may determine whether biometric data and/or sensor data is missing based on data, for example, biometric data and/or sensor data, provided from the generative AI application 310 and may generate missing biometric data and/or missing sensor data.

According to an embodiment, the specified-domain AI model 331 may include a specified-domain, for example, health-based, artificial intelligence model. For example, the specified-domain AI model 331 may include a model personalized for a domain and a model used when processing data requiring security, such as biometric data of the user.

For example, the general AI model 333 may include an artificial intelligence model for collecting, training, analyzing, and/or predicting data regardless of a specific purpose and/or domain. The general AI model 333 may include an artificial intelligence model applicable to all data rather than to a specific entity. For example, the general AI model 333 may include, but is not limited to, a large language model (LLM), a generative adversarial network, and/or a variational autoencoder.

According to an embodiment, the hardware 350 may include at least one processing device, for example, an NPU, a GPU, or a CPU, and at least one hardware component, for example, at least one sensor.

FIG. 4 is a drawing for describing an operation of generating missing biometric data in an electronic device according to an embodiment.

According to an embodiment, an electronic device, for example, the electronic device 100 of FIG. 1, the electronic device 210 of FIG. 2, or the electronic device 1501 of FIG. 15, may generate missing biometric data, for example, generative data 440, using an AI model based on measured biometric data, hereinafter referred to as “activity data/health data 410,” and/or text data 420.

According to an embodiment, the electronic device may obtain the activity data/health data 410, which may be referred to as biometric data in the disclosure, using a sensor of the electronic device and/or a sensor of an external electronic device, for example, a wearable device. The activity data/health data 410 may include data corresponding to a raw signal measured using at least one sensor and may include quantitative and/or qualitative data generated based on the raw signal. For example, the activity data/health data 410 may include action tracking data, stress tracking data, sleep tracking data, step tracking data, HRV data, mind tracking data, location data, and a user profile. For example, the activity data/health data 410 is not limited to the items illustrated in FIG. 4. For example, the action tracking data may include information about an action, for example, a degree, continuity, strength, and/or a displacement difference of an action, per specified time, for example, per minute, of the user. The stress tracking data may include information about a stress index and/or a stress type, for example, acute stress or chronic stress, of the user. The sleep tracking data may include information about a sleep state, a sleep time, and/or a sleep pattern of the user. The step tracking data may include information about a walking pattern of the user, a step count of the user, a state in which the user walks or runs, and/or a movement speed of the user. The HRV data may include information associated with heart rate of the user and/or regularity of heartbeat. The mind tracking data may include information associated with a depression index of the user. The location data may include information about a location and/or a movement trajectory of the electronic device. The user profile may include information associated with personal information of the user, for example, a health state, body information, a name, a gender, an age, a preference of the user, activity history, and/or device usage history.

According to an embodiment, the AI model 430, for example, the AI model 213 of FIG. 2, the AI model 330 of FIG. 3, or the AI model 930 of FIG. 9, may receive the activity data/health data 410 and may generate missing data. For example, a specified-domain AI model 431, for example, a health AI model, may generate or estimate new data based on data associated with a specified domain, for example, a health domain.

According to an embodiment, the specified-domain AI model 431, for example, the specified-domain AI model 931 of FIG. 9, may estimate a new index based on the biometric data 410, for example, the activity data/health data 410. For example, the specified-domain AI model 431 may analyze a sleep type of the user based on accumulated sleep tracking data of the user or may measure a menstrual cycle of a female user based on accumulated skin temperature information and/or heart rate information of the user.

According to an embodiment, the specified-domain AI model 431 may generate missing data. For example, when there is a day, hereinafter referred to as a “missing day,” on which the activity data/health data 410 associated with activity of the user is missing due to deactivation or malfunction of a sensor and/or because the user does not have the electronic device or an external electronic device, the specified-domain AI model 431 may generate missing activity data/health data 410 based on a history of the activity data/health data 410 measured on another day other than the missing day. According to an embodiment, when the missing activity data/health data 410 cannot be generated based on the activity data/health data 410 measured on another day or when accuracy is to be increased, the electronic device may receive the text data 420. For example, the text data 420 may include information associated with behavior of the user on the missing day. For example, it may be assumed that the electronic device receives, from the user, a voice input or text input stating, “I went to the supermarket to buy groceries at lunchtime and went for a run around the park late at night.” The AI model 430 may generate a plurality of tokens based on a semantic unit of text, such as a text input represented as “went to-supermarket-buy-groceries-at-lunchtime-went-run-around-park-late-night.” For example, the AI model 430 may generate the plurality of tokens from the voice input or the text input based on a semantic unit of each language, depending on the language. For example, for an English voice input or text input, the AI model 430 may generate each word of the input as a token. For example, the AI model 430 may generate the plurality of tokens based on different semantic units depending on a scale, a characteristic, a training scheme, training data, and/or a type of the AI model 430. For example, the AI model 430, for example, the specified-domain AI model 431, may perform prompting with the general AI model 433, for example, the general AI model 933 of FIG. 9, based on the tokens from the text input to output data in a form corresponding to previously recorded activity data/health data 410. For example, the general AI model 433 may output data in the same form as the previously recorded activity data/health data 410 based on the tokens. For example, the general AI model 433 may extract or infer main keyword information from the tokens, such as “lunch is from 11:00 to 13:00,” “mart is location information,” “late at night is after 22:00,” and “running is movement at a speed of 4 km/h or more.” The general AI model 433 may provide the extracted or inferred information to the specified-domain AI model 431. The specified-domain AI model 431 may determine similarity to existing activity data/health data 410 based on the information provided from the general AI model 433 and may generate missing activity data/health data 410 based thereon. For example, when the missing day is yesterday, the specified-domain AI model 431 may extract data and/or information estimated as similar to yesterday and necessary to generate data for the missing day based on the activity data/health data 410 except for the missing day and/or the information received from the general AI model 433, and may generate the missing activity data/health data based on the extracted data and/or information.

According to an embodiment, when there is no sensor data or insufficient sensor data for generating the activity data/health data 410, the AI model 430, for example, the specified-domain AI model 431, may generate required sensor data. For example, when measuring daily calorie consumption of the user, a calculated calorie-consumption value may vary depending on movement speed, a gradient, for example, uphill or downhill, or an activity type, for example, walking or running. For example, even when the same step count is measured, calorie consumption may increase more for an uphill movement than for a downhill movement. In this case, the AI model 430 may generate altitude data estimated based on pedometer information for measuring a step count of the user and location information of the electronic device, for example, a mountain location. When calculating daily calorie consumption, the AI model 430 may additionally consider the generated altitude data to calculate and/or modify the calorie consumption.

According to an embodiment, FIG. 4 illustrates a step count, an activity time, and calories burned as the generative data 440, but the generative data 440 is not limited thereto. Any of the missing biometric data 410, for example, the activity data/health data 410, may be provided as the generative data 440.

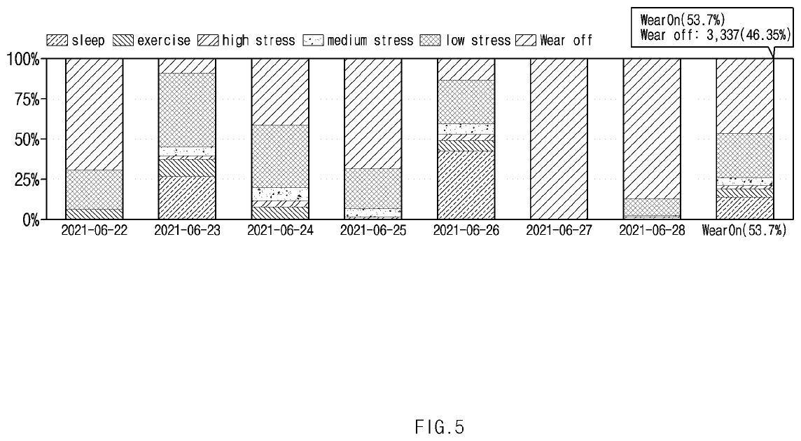

FIG. 5 is a drawing for describing an operation of detecting that data is missing in an electronic device according to an embodiment. For example, the description of FIG. 5 assumes that an electronic device, for example, the electronic device 100 of FIG. 1, the electronic device 210 of FIG. 2, or the electronic device 1501 of FIG. 15, obtains biometric data and/or sensor data from an external electronic device, for example, a wearable device such as the wearable device 220 of FIG. 2.

According to an embodiment, the electronic device may identify biometric information measured for each specified cycle, for example, a daily cycle, although embodiments are not limited thereto. For example, FIG. 5 is a graph illustrating day-to-day biometric information, for example, sleep, exercise, high stress, medium stress, low stress, and wear-off information, identified by the electronic device. According to various embodiments, biometric information is not limited to that illustrated and described with reference to FIG. 5. For example, the electronic device may determine whether biometric data is missing based on a wear-off ratio for each specified period, for example, each day. For example, when a time during which the external electronic device is not worn exceeds a specified threshold time, the electronic device may determine that the biometric data is missing during the corresponding period. For example, when obtaining biometric data from the external electronic device, the electronic device may fail to obtain the biometric data during a period in which the user does not wear the external electronic device. For example, when a time during which the external electronic device is not worn during a specified period, for example, one day or 24 hours, exceeds the specified threshold time, for example, 8 hours, or a specified threshold ratio, for example, about 70%, the electronic device may determine that the biometric data is missing on the corresponding date. Referring to FIG. 5, for example, when the specified threshold time is 12 hours, that is, the threshold ratio is 50%, the electronic device may determine that the biometric data is missing on 2021 Jun. 22, 2021 Jun. 25, 2021 Jun. 27, and 2021 Jun. 28.

According to an embodiment, the electronic device may compare a time during which the biometric data is missing with the specified threshold time based at least in part on an amount of time during which the biometric data or sensor data is measured and/or an operation state of the electronic device, and may determine that the biometric data or sensor data is missing when the time exceeds the specified threshold time. For example, when a ratio between a time during which the biometric data or sensor data is not obtained for each specified cycle or specified session and a total duration of the specified cycle or specified session exceeds the specified threshold, the electronic device may determine that the biometric data or sensor data is missing in the corresponding period or session.

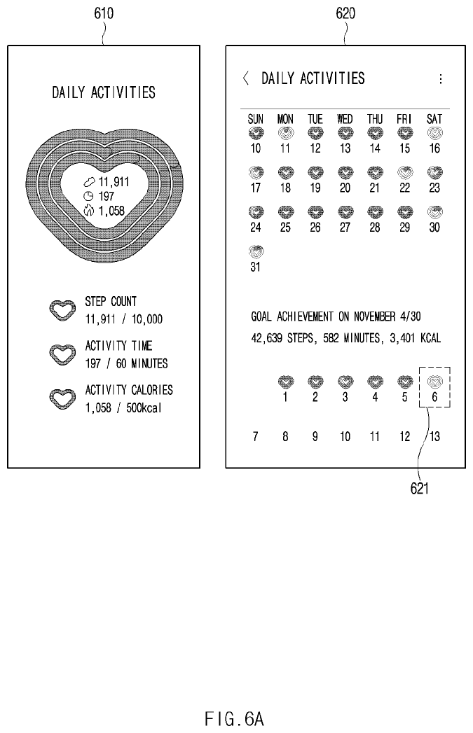

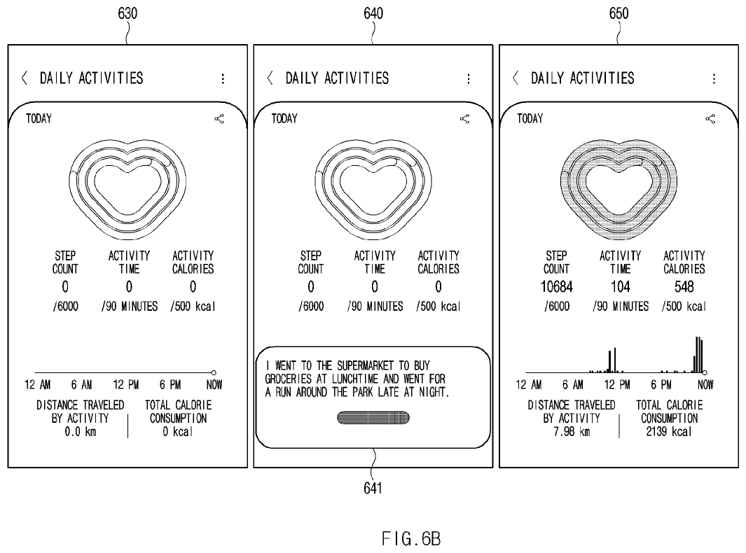

FIGS. 6A and 6B are drawings for describing an operation of generating and providing missing data in an electronic device according to an embodiment.

According to an embodiment, an electronic device, for example, the electronic device 100 of FIG. 1, the electronic device 210 of FIG. 2, or the electronic device 1501 of FIG. 15, may provide a user interface including measured biometric data, for example, activity data/health data 410 of FIG. 4, and information indicating whether the biometric data is missing. For example, the description of FIGS. 6A and 6B assumes that a specified period is a daily unit, but embodiments are not limited thereto.

For example, a first screen 610 may be a screen displaying biometric information of a user in a current cycle, for example, today. For example, the first screen 610 may include an image and/or information indicating an amount of daily activity of the user. For example, the first screen 610 may include information indicating a step count/target step count, an activity time/target activity time, and activity calories/target calorie consumption of the user and a visual element indicating whether a target value has been achieved.

For example, a second screen 620 may summarize and display biometric information of the user measured for each cycle. For example, the second screen 620 may summarize and provide biometric information for each specified cycle, for example, each day, over a period, for example, a month, longer than the specified cycle. For example, the second screen 620 may include a visual element 621 indicating a degree to which biometric information for each cycle is measured. For example, the visual element 621 may have a form with an unfilled interior to indicate that biometric information is missing on a corresponding date. For example, FIG. 6A illustrates indicating a degree to which biometric information is measured according to a degree to which the interior of a figure is filled with another color or that the biometric information is missing, but the display method is not limited thereto. For example, the visual element may indicate a degree to which biometric information is measured according to color, size, shape, and/or contrast, or may indicate that the biometric information is missing.

For example, a third screen 630 may display biometric information for a date on which biometric data is missing. For example, the electronic device may receive, through a user interface, a user input selecting a visual element, for example, the visual element 621, indicating that biometric data is missing. The electronic device may display the third screen 630 including biometric information during a cycle, that is, a date, selected according to the user input. For example, because the biometric data is missing, all values indicating the biometric data on the third screen 630 may be set to initial values.

For example, in response to outputting the third screen 630, the electronic device may output, through the user interface, a message requesting information associated with behavior of the user during the period or date in which the biometric data is missing.

For example, a fourth screen 640 may display content of a user input in a region 641 when the electronic device receives the user input including information associated with behavior of a user during a cycle or date in which biometric data is missing. For example, the region 641 of the user interface may display text content identified from the user input. For example, the user input may include a voice input and/or an input received through the user interface.

According to an embodiment, a fifth screen 650 may be a screen on which the electronic device generates and displays biometric data during the period or date using a generative artificial intelligence model, for example, the AI model 213 of FIG. 2, the AI model 330 of FIG. 3, the AI model 430 of FIG. 4, or the AI model 930 of FIG. 9. For example, the electronic device may generate missing biometric data based on biometric data obtained on days other than the missing day and/or the user input using the generative artificial intelligence model. The fifth screen 650 may include information and a visual element indicating the generated biometric data. The fifth screen 650 may include information and/or a diagram analyzed using the generated biometric data.

According to various embodiments, the configuration of the user interface provided by the electronic device and the information and/or visual elements included in the user interface are not limited to those illustrated in FIG. 6.

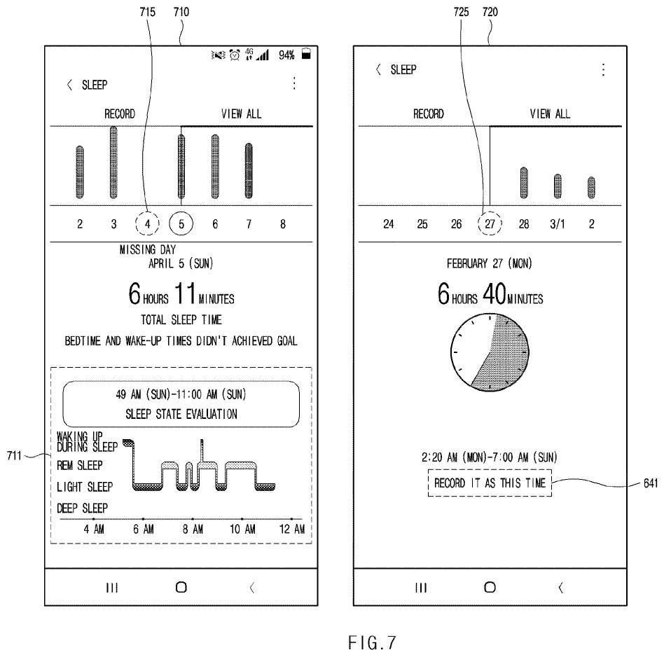

FIG. 7 is a drawing for describing an operation of generating and providing missing data in an electronic device according to an embodiment. For example, FIG. 7 illustrates an example of a user interface screen for providing sleep data among biometric data.

According to an embodiment, an electronic device, for example, the electronic device 100 of FIG. 1, the electronic device 210 of FIG. 2, or the electronic device 1501 of FIG. 15, may provide biometric data through a user interface. A first screen 710 of the user interface may include a graph indicating sleep data measured for each day and detailed information of the sleep data for each day, for example, total sleep time and information obtained by analyzing a sleep state. For example, the first screen 710 may indicate that sleep data is missing 715 for a specific cycle or date, for example, April 4, through the graph indicating the sleep data measured for each day. For example, the electronic device may generate and provide missing sleep data based on biometric data measured on dates other than the missing day, for example, April 4, and/or a user input using a generative artificial intelligence model, for example, the AI model 213 of FIG. 2, the AI model 330 of FIG. 3, the AI model 430 of FIG. 4, or the AI model 930 of FIG. 9.

According to an embodiment, a second screen 720 may be a screen on which the user interface provides an item for generating missing sleep data corresponding to a missing day, for example, February 27, and for allowing a user to confirm information of the generated sleep data. For example, the second screen 720 may indicate that sleep data is missing 725 for a specific cycle or date, for example, February 27, through a graph indicating sleep data measured for each day. For example, the second screen 720 may provide information of the generated sleep data, for example, a time at which the user falls asleep, a time at which the user awakes, and a sleep time, and may receive an input for confirming whether to store the generated biometric data from the user through the item 641. For example, when receiving the input for storing the generated biometric data from the user, the electronic device may store the generated biometric data. When the input for storing the generated missing biometric data is not received from the user, the electronic device may refrain from storing the generated missing biometric data.

According to various embodiments, the configuration of the user interface provided by the electronic device and the information and/or visual elements included in the user interface are not limited to those illustrated in FIG. 7.

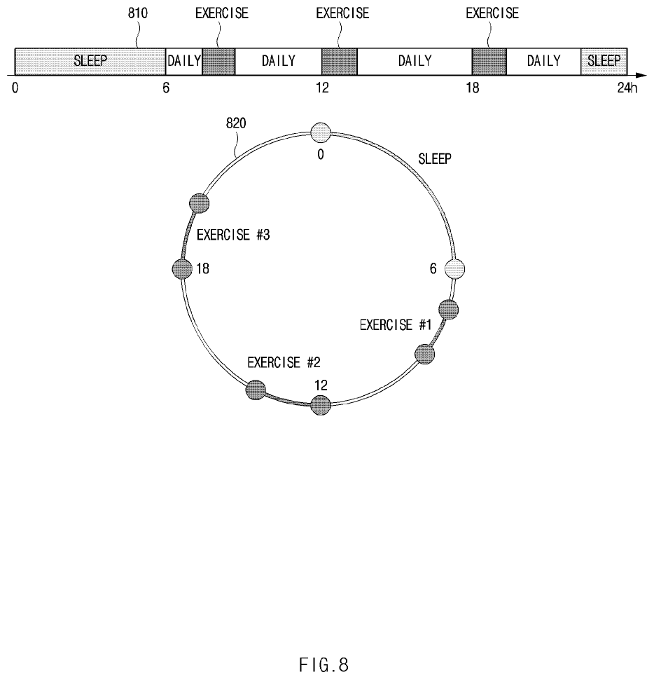

FIG. 8 illustrates an example of a user interface provided to manage data by an electronic device according to an embodiment.

According to an embodiment, the electronic device may provide interfaces 810 and 820 for modifying information of biometric data corresponding to one cycle, for example, a specific date, and the biometric data.

For example, the interfaces 810 and 820 may provide information of the biometric data in the form of a specified graph. For example, FIG. 8 illustrates a case in which the interfaces 810 and 820 include a graph indicating an activity state, for example, sleep, daily life, or exercise, of a user during one cycle, for example, one day, of the user. For example, the interfaces may include a first graph 810 indicating a daily activity state of the user and a second graph 820 configured to adjust a time of each activity state through a user input. For example, the second graph 820 may be provided in the form of a circular graph capable of selecting start points and end points of respective activity states of the user for each day. For example, the user may select a point indicating a start point or an end point of an activity state and may input a gesture, for example, a drag input in a specific direction, to adjust a start time or an end time of the activity state. According to an embodiment, when a portion of the biometric data is modified by the user input, the electronic device may store the modified biometric data.

FIG. 8 illustrates an example of an interface capable of displaying and adjusting biometric data. Interfaces provided by the electronic device are not limited to those illustrated in FIG. 8.

FIG. 9 is a drawing for describing an operation of generating missing sensor data in an electronic device according to an embodiment. Hereinafter, the same or similar description as that of FIG. 4 may be omitted or briefly described.

According to an embodiment, an electronic device, for example, the electronic device 100 of FIG. 1, the electronic device 210 of FIG. 2, or the electronic device 1501 of FIG. 15, may generate missing data, that is, generative data 940, using an AI model 930, for example, the AI model 213 of FIG. 2, the AI model 330 of FIG. 3, or the AI model 430 of FIG. 4, based on measured sensor data 910 and/or text data 920.

According to an embodiment, the electronic device may obtain the sensor data 910 using a sensor of the electronic device and/or a sensor of an external electronic device, for example, a wearable device. The sensor data 910 may include data corresponding to a raw signal measured using at least one sensor. For example, the sensor data 910 may include electrocardiogram data, galvanic skin response data, electroencephalogram data, bioelectrical impedance analysis data, photoplethysmography data, accelerometer data, gyroscope data, temperature data, time-of-flight data, ultra-wideband data, GPS data, pedometer data, barometer data, and a user profile. For example, the sensor data 910 is not limited to those illustrated in FIG. 9.

According to an embodiment, the AI model 930 may receive the sensor data 910 to generate missing data, that is, generative data 940. According to an embodiment, a specified-domain AI model 931 may generate or estimate new data based on data associated with a specified domain, for example, a health domain.

According to an embodiment, the specified-domain AI model 931 may estimate or generate new sensor data 910 based on the measured sensor data 910. For example, the specified-domain AI model 931 may generate missing data. For example, when there is a session, hereinafter referred to as a “missing session,” in which the sensor data 910 is missing due to deactivation or malfunction of at least one sensor and/or because the user does not have the electronic device or an external electronic device, the specified-domain AI model 931 May generate missing sensor data 940, that is, the generative data 940, based on a history of the sensor data 910 measured using another sensor other than the sensor corresponding to the missing sensor data 940 during the missing session. The specified-domain AI model 931 may generate the missing sensor data 940 based on a history of the sensor data 910 measured during another session different from the missing session. For example, the specified-domain AI model 931 may generate the missing sensor data 940 based on sensor data 910 measured from a sensor other than a first sensor corresponding to the missing sensor data 940 in the same session, that is, the missing session, and/or sensor data 910 measured from the first sensor or another sensor in a different session.

According to an embodiment, when the missing sensor data 940 cannot be generated based on the sensor data 910 measured using another sensor during the missing session and/or the sensor data 910 measured during another session other than the missing session, or when accuracy is to be increased, the electronic device may receive text data 920 from the user. For example, the text data 920 may include information associated with behavior of the user in the missing session. For example, it may be assumed that the electronic device receives a voice input or text input stating, “I ran a 400 m track for 5 laps at a pace of 1 minute and 30 seconds.” The AI model 930 may generate a plurality of tokens based on a semantic unit of text, such as a text input represented as “ran-400-m-track-5-laps-at-pace-1 minute-30 seconds.” For example, the AI model 930, for example, the specified-domain AI model 931, may perform prompting with a general AI model 933 based on tokens from the text input to output data in a form corresponding to previously recorded sensor data 910. For example, the general AI model 933 may output data in the same form as the previously recorded sensor data 910 based on the tokens. For example, the general AI model 933 may extract or infer main keyword information from the tokens. The general AI model 933 may provide the extracted or inferred information to the specified-domain AI model 931. The specified-domain AI model 931 may determine similarity to existing sensor data 910 based on the information provided from the general AI model 933 and may generate the missing sensor data 940 based thereon.

According to an embodiment, FIG. 9 illustrates PPG data, accelerometer data, atmospheric pressure data, and gyroscope data as the generative data 940, but the generative data 940 is not limited thereto. Any of the missing sensor data 940 may be provided as the generative data 940.

For example, the electronic device, for example, a mobile terminal, may obtain biometric data and/or sensor data of the user using at least one sensor or may obtain biometric data and/or sensor data of the user, measured by an external electronic device, for example, a wearable device, from the external electronic device. In some cases, the user may need to continuously carry or wear the electronic device or the external electronic device such that the electronic device may obtain biometric data and/or sensor data of the user. For example, when the user does not continue carrying or wearing the electronic device or the external electronic device, biometric data and/or sensor data may be missing. In this case, biometric information and/or health information of the user may fail to be accurately provided, bias may occur, and an error may occur in data statistics. Furthermore, it may take a relatively large amount of time and effort to obtain biometric data and/or sensor data sufficient to analyze a health state of the user. According to embodiments of the disclosure, an electronic device capable of generating missing biometric data and/or sensor data using a generative artificial intelligence model to supplement required data even when biometric data and/or sensor data is missing, a method, and a storage medium are provided.

According to an embodiment, an electronic device may include communication circuitry, a display, at least one sensor, a memory including a generative artificial intelligence model, and a processor. According to an embodiment, the memory may store instructions that, when executed by the processor, control an operation of the electronic device.

According to an embodiment, the instructions, when executed by the processor, may cause the electronic device to identify biometric data periodically measured using the at least one sensor.

According to an embodiment, the instructions, when executed by the processor, may cause the electronic device to determine whether the biometric data is missing. For example, the instructions, when executed by the processor, may cause the electronic device to determine whether the biometric data is missing based on the identifying of the biometric data of the user.

According to an embodiment, the instructions, when executed by the processor, may cause the electronic device to identify information related to a period during which the biometric data is missing.

According to an embodiment, the instructions, when executed by the processor, may cause the electronic device to identify a measurement history of previously measured biometric data.

According to an embodiment, the instructions, when executed by the processor, may cause the electronic device to generate, using the generative artificial intelligence model, missing biometric data based at least in part on the measurement history.

According to an embodiment, the instructions, when executed by the processor, may cause the electronic device to receive text data associated with behavior of a user during the period.

According to an embodiment, the instructions, when executed by the processor, may cause the electronic device to generate the missing biometric data based on the text data and the measurement history.

According to an embodiment, the at least one sensor may include a plurality of sensors.

According to an embodiment, the instructions, when executed by the processor, may cause the electronic device to identify sensor data measured using the plurality of sensors during a specified period.

According to an embodiment, the instructions, when executed by the processor, may cause the electronic device to determine whether sensor data corresponding to at least one sensor among the plurality of sensors is missing.

According to an embodiment, the instructions, when executed by the processor, may cause the electronic device to identify measured sensor data excluding missing sensor data during the specified period.

According to an embodiment, the instructions, when executed by the processor, may cause the electronic device to generate, using the generative artificial intelligence model, the missing sensor data based at least in part on the measured sensor data.

According to an embodiment, the instructions, when executed by the processor, may cause the electronic device to identify a measurement history of sensor data measured in a previous measurement period corresponding to the specified period.

According to an embodiment, the instructions, when executed by the processor, may cause the electronic device to generate the missing sensor data based at least in part on the measurement history.

According to an embodiment, the instructions, when executed by the processor, may cause the electronic device to receive text data associated with behavior of a user during the specified period from the user.

According to an embodiment, the instructions, when executed by the processor, may cause the electronic device to generate the missing sensor data based at least in part on the text data.

According to an embodiment, the instructions, when executed by the processor, may cause the electronic device to obtain at least some of biometric data or sensor data obtained through a sensor of an external electronic device from the external electronic device.

According to an embodiment, the instructions, when executed by the processor, may cause the electronic device to determine whether data is missing for the obtained biometric data or sensor data.

According to an embodiment, the instructions, when executed by the processor, may cause the electronic device to generate, using the generative artificial intelligence model, missing data for the obtained biometric data or sensor data.

According to an embodiment, the instructions, when executed by the processor, may cause the electronic device to compare, for each specified cycle, a time during which the biometric data is missing with a specified threshold time based on at least one of an amount of time during which the biometric data is measured or an operation state of the electronic device.

According to an embodiment, the instructions, when executed by the processor, may cause the electronic device to determine that the biometric data is missing based on the time exceeds the specified threshold time.

According to an embodiment, the instructions, when executed by the processor, may cause the electronic device to output, through the display, a user interface including the measured biometric data and information indicating whether the biometric data is missing.

According to an embodiment, the instructions, when executed by the processor, may cause the electronic device to receive, through the user interface, a user input confirming whether to use the generated missing biometric data based on the missing biometric data is generated.

According to an embodiment, the instructions, when executed by the processor, may cause the electronic device to store the generated missing biometric data in the memory based on the user input.

According to an embodiment, the instructions, when executed by the processor, may cause the electronic device to receive a user input for modifying the generated missing biometric data or the generated missing sensor data.

According to an embodiment, the instructions, when executed by the processor, may cause the electronic device to store the modified missing biometric data or modified missing sensor data in the memory based on the user input.

According to an embodiment, the instructions, when executed by the processor, may cause the electronic device to obtain sensor data measured using the at least one sensor.

According to an embodiment, the instructions, when executed by the processor, May cause the electronic device to set a parameter corresponding to a specified condition.

According to an embodiment, the instructions, when executed by the processor, may cause the electronic device to generate, using the generative artificial intelligence model, sensor data under the specified condition based on the parameter and the obtained sensor data.

According to embodiments of the disclosure, biometric data and/or sensor data missing during a specified cycle or a specified period may be identified, and missing biometric data and/or missing sensor data may be generated and used using the generative artificial intelligence model. According to embodiments of the disclosure, sensor data corresponding to a virtual condition may be generated in various ways, thereby generating sensor data usable to develop, analyze, and/or evaluate an algorithm even in a limited environment.

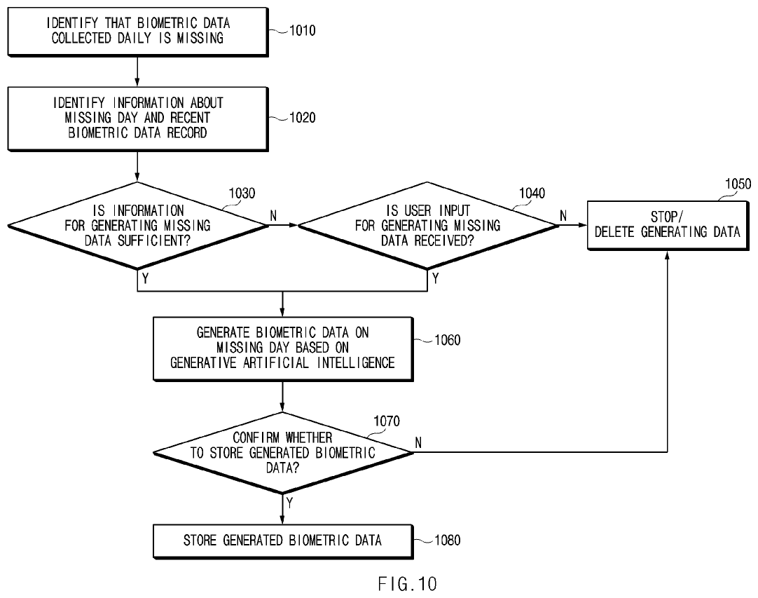

FIG. 10 is a flowchart of a method for managing data of an electronic device according to an embodiment.

In an embodiment described below, respective operations may be sequentially performed, but are not necessarily limited to sequential performance. For example, an order of the respective operations may be changed, and at least two operations may be performed in parallel.

According to an embodiment, operations 1010 to 1080 may be understood as being performed by a processor, for example, the processor 150 of FIG. 1, of an electronic device, for example, the electronic device 100 of FIG. 1.

According to an embodiment, in operation 1010, an electronic device, for example, the electronic device 100 of FIG. 1, the electronic device 210 of FIG. 2, or the electronic device 1501 of FIG. 15, may identify that biometric data collected daily is missing. For example, the electronic device may identify a day on which biometric data is not collected or measured. According to an embodiment, the electronic device may receive biometric data obtained by an external electronic device from the external electronic device. The electronic device may identify whether at least a portion of the biometric data collected daily is missing based on the biometric data received from the external electronic device. According to an embodiment, the electronic device may determine whether biometric data is missing based on information indicating a state in which the external electronic device, for example, a wearable device, is worn. For example, the electronic device may determine whether biometric data is missing based on information about a time during which the user wears the external electronic device or a time during which the user does not wear the external electronic device. For example, the electronic device may determine whether a daily wear time of the external electronic device is less than a specified threshold time and may determine that biometric data is missing on a day for which the wear time is less than the threshold time. In FIG. 10, the description is provided assuming that the cycle is one day, but embodiments are not limited thereto. The cycle may be specified as various periods. For example, the electronic device may identify that biometric data collected weekly is missing or may identify that biometric data collected monthly is missing.

According to an embodiment, in operation 1020, the electronic device may identify information about a missing day and a recent biometric data record. For example, the electronic device may identify the missing day and information associated with the missing day, for example, weather, a day of the week, a date, that is, a day, a month, and/or a year, and/or schedule information of the corresponding date. The electronic device may identify a record of biometric data measured before and after the missing day.

According to an embodiment, in operation 1030, the electronic device may determine whether information for generating missing biometric data is sufficient. For example, the electronic device may determine whether missing biometric data can be generated based on the information about the missing day and the recent biometric data record identified in operation 1020. According to an embodiment, the electronic device may perform operation 1060 when the information for generating the missing data is sufficient and may perform operation 1040 when the information is not sufficient.

According to an embodiment, in operation 1040, the electronic device may identify whether a user input for generating missing data has been received. According to an embodiment, the electronic device may output a message requesting the user input to generate the missing data. For example, the electronic device may output a message requesting confirmation of whether there was specific behavior by the user on the missing day, such as “Did you go to work on the missing day?” or “Did you do your usual exercise from 8 to 10 on the missing day?” According to an embodiment, the user input may include information associated with behavior performed by the user on the missing day. For example, the user input may include a text input associated with the behavior of the user on the missing day. For example, the electronic device may receive, from the user, a text input associated with behavior on the missing day, such as “I woke up at 6:00 on the missing day, went to work for 30 minutes starting at 8:00, took a walk for about 30 minutes at 12:00, got off work at 6:00, and went to sleep around 10:00.” For example, the text input may include a voice input of the user and/or an input through a user interface.

According to an embodiment, the electronic device may perform operation 1060 when receiving the user input for generating the missing data and may perform operation 1050 when the user input is not received.

According to an embodiment, in operation 1050, the electronic device may stop or delete generation of the missing data. For example, when the information for generating the missing data is not sufficient and the user input is not received, the electronic device may stop generating the missing data and may provide a notification indicating that the missing data cannot be generated. For example, when the user confirms that the generated missing biometric data is not to be stored, the electronic device may delete the generated missing biometric data.

According to an embodiment, in operation 1060, the electronic device may generate biometric data for the missing day based on a generative artificial intelligence model, for example, the AI model 213 of FIG. 2, the AI model 330 of FIG. 3, the AI model 430 of FIG. 4, or the AI model 930 of FIG. 9. For example, the electronic device may generate missing biometric data based on the recent biometric data record and/or the user input using the generative artificial intelligence model. For example, the electronic device may generate missing biometric data based on biometric data previously measured on a date having an environment similar to that of the missing day and/or information associated with behavior of the user on the missing day.

According to an embodiment, in operation 1070, the electronic device may confirm whether to store the generated missing biometric data. For example, the electronic device may provide a user interface for confirming whether to use the generated missing biometric data. The electronic device may receive, through the user interface, a user input for determining whether to use or store the generated missing biometric data. According to an embodiment, the electronic device may perform operation 1080 when receiving the user input indicating that the generated missing biometric data is to be stored and may perform operation 1050 when receiving the user input indicating that the generated missing biometric data is not to be stored.

According to an embodiment, in operation 1080, the electronic device may store the generated missing biometric data in a memory.

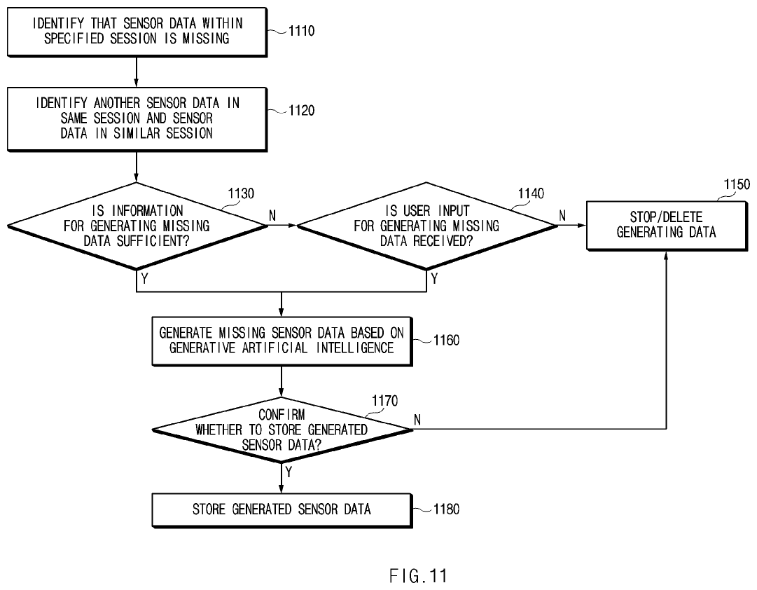

According to various embodiments, an order of the operations of FIG. 10 may be changed, at least some of the operations may be omitted, and/or at least one operation, for example, at least one of the operations of FIGS. 11 to 14, may be added.

According to an embodiment, biometric data missing during a specified cycle may be identified, and missing biometric data may be generated and used using the generative artificial intelligence model.

FIG. 11 is a flowchart of a method for managing data of an electronic device according to an embodiment.

In an embodiment described below, respective operations may be sequentially performed, but are not necessarily limited to sequential performance. For example, an order of the respective operations may be changed, and at least two operations may be performed in parallel.

According to an embodiment, operations 1110 to 1180 may be understood as being performed by a processor, for example, the processor 150 of FIG. 1, of an electronic device, for example, the electronic device 100 of FIG. 1.

According to an embodiment, in operation 1110, an electronic device, for example, the electronic device 100 of FIG. 1, the electronic device 210 of FIG. 2, or the electronic device 1501 of FIG. 15, may identify that sensor data within a specified session is missing. For example, the electronic device may detect a case in which at least one sensor is deactivated within the specified session and sensor data is not obtained. For example, when obtaining sensor data using a plurality of sensors, the electronic device may identify a case in which sensor data corresponding to the at least one sensor is not measured. According to an embodiment, the plurality of sensors may include at least one sensor included in the electronic device and/or at least one sensor included in an external electronic device. For example, the electronic device may detect not only a case in which the sensor data is not collected at all but also a case in which quality of particular sensor data is poor or inaccurate or a case in which an error is determined to exist in sensor data, for example, a case in which the sensor data is out of a specified range. According to an embodiment, a session may refer to a time within a certain period specified by the electronic device.

According to an embodiment, in operation 1120, the electronic device may identify other sensor data in the same session and sensor data in a similar session. For example, the electronic device may identify sensor data obtained using another sensor other than a sensor corresponding to the missing sensor data. The electronic device may determine a previous session similar to a session in which sensor data is missing based on session-related information, for example, a period, a date, a time zone, a day of the week, weather, and/or a season. The electronic device may identify sensor data measured in the similar previous session.

According to an embodiment, in operation 1130, the electronic device may determine whether information for generating missing data is sufficient. For example, the electronic device may determine whether missing sensor data can be generated based on the other sensor data in the same session and the sensor data in the similar session identified in operation 1120. According to an embodiment, the electronic device may perform operation 1160 when the information for generating the missing data is sufficient and may perform operation 1140 when the information is not sufficient.

According to an embodiment, in operation 1140, the electronic device may identify whether a user input for generating missing data has been received. According to an embodiment, the electronic device may output a message requesting the user input to generate the missing data. According to an embodiment, the electronic device may receive, from the user, a user input including information associated with behavior of the user within the session. For example, the user input may include a text input associated with behavior of the user during the session. For example, the electronic device may receive a text input such as “I ran a 400 m track for 5 laps at a pace of 1 minute and 30 seconds.” or “I felt my heart pounding while eating dinner.” For example, the text input may include a voice input of the user and/or an input through a user interface.

According to an embodiment, the electronic device may perform operation 1160 when receiving the user input for generating the missing data and may perform operation 1150 when the user input is not received.

According to an embodiment, in operation 1150, the electronic device may stop or delete generation of the missing data. For example, when the information for generating the missing data is not sufficient and the user input is not received, the electronic device may stop generating the missing data and may provide a notification indicating that the missing data cannot be generated. For example, when the user confirms that the generated missing sensor data is not to be stored, the electronic device may delete the generated missing sensor data.

According to an embodiment, in operation 1160, the electronic device may generate missing sensor data within the session based on a generative artificial intelligence model, for example, the AI model 213 of FIG. 2, the AI model 330 of FIG. 3, the AI model 430 of FIG. 4, or the AI model 930 of FIG. 9. For example, the electronic device may generate missing sensor data based on the other sensor data in the same session, the sensor data in the similar session, and/or the user input using the generative artificial intelligence model. For example, it may be assumed that the electronic device obtains sensor data using a PPG sensor, for example, for heart-rate measurement, a barometer, for example, for altitude measurement, and a pedometer, for example, for average-speed measurement, to measure an exercise state of the user in a specific session. For example, when detecting that sensor data of one of the PPG sensor, the barometer, or the pedometer is missing in the same session, the electronic device may generate missing sensor data based on sensor data obtained using the remaining two sensors in the same session using the generative artificial intelligence model. For example, when there is sensor data measured using the PPG sensor, the barometer, and/or the pedometer in another similar session, the electronic device may generate missing sensor data based on sensor data in the other similar session. For example, the electronic device may generate missing sensor data based on sensor data measured in a situation or session associated with behavior of the user, based on the user input including the information associated with the behavior of the user in the corresponding session.

According to an embodiment, in operation 1170, the electronic device may confirm whether to store the generated missing sensor data. For example, the electronic device may provide a user interface for confirming whether to use the generated missing sensor data. The electronic device may receive, through the user interface, a user input for determining whether to use or store the generated missing sensor data. According to an embodiment, the electronic device may perform operation 1180 when receiving the user input indicating that the generated missing sensor data is to be stored and may perform operation 1150 when receiving the user input indicating that the generated missing sensor data is not to be stored.

According to an embodiment, in operation 1180, the electronic device may store the generated missing sensor data in a memory.

According to various embodiments, an order of the operations of FIG. 11 may be changed, at least some of the operations may be omitted, and/or at least one operation, for example, at least one of the operations of FIGS. 10 and 12 to 14, may be added.

According to an embodiment, sensor data missing during a specified cycle may be identified, and missing sensor data may be generated and used using the generative artificial intelligence model.

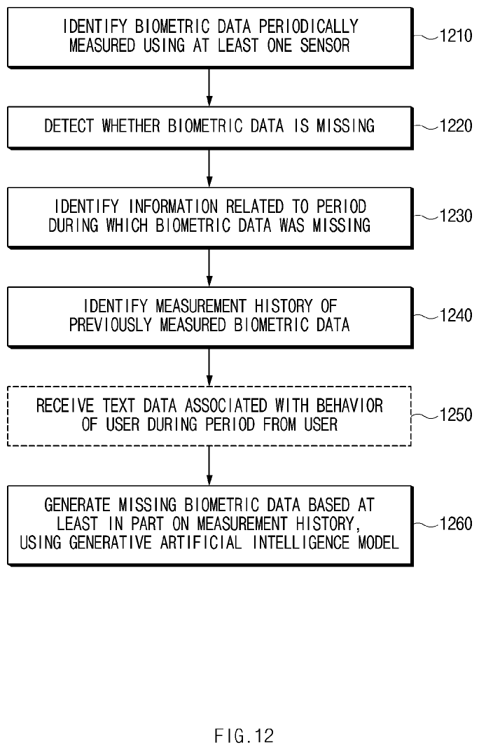

FIG. 12 is a flowchart of a method for managing data of an electronic device according to an embodiment.

In an embodiment described below, respective operations may be sequentially performed, but are not necessarily limited to sequential performance. For example, an order of the respective operations may be changed, and at least two operations may be performed in parallel.

According to an embodiment, operations 1210 to 1260 may be understood as being performed by a processor, for example, the processor 150 of FIG. 1, of an electronic device, for example, the electronic device 100 of FIG. 1.

According to an embodiment, in operation 1210, an electronic device, for example, the electronic device 100 of FIG. 1, the electronic device 210 of FIG. 2, or the electronic device 1501 of FIG. 15, may identify biometric data periodically measured using at least one sensor. For example, a measurement cycle of the biometric data may include, but is not limited to, one day, one month, or one year, and may be specified as any period. According to an embodiment, the electronic device may receive biometric data measured using a sensor of an external electronic device, for example, a wearable device, from the external electronic device.

According to an embodiment, in operation 1220, the electronic device may determine whether the biometric data is missing. For example, when the period is one day, the processor 150 may identify a day on which the biometric data is missing, hereinafter referred to as a “missing day.” According to an embodiment, the processor 150 may determine whether the biometric data is missing based on a result of comparing, for each specified cycle, a time at which the biometric data is measured, an operation state of the electronic device, for example, a time at which the electronic device is powered on, a time at which the electronic device is powered off, or a time at which a sensor is activated or deactivated, or a time during which the external electronic device, for example, the wearable device, is worn, with a specified threshold, for example, a threshold time.

According to an embodiment, in operation 1230, the electronic device may identify information related to a period during which the biometric data is missing. For example, the electronic device may identify information associated with the missing day of the biometric data, for example, a date, weather, a day of the week, and/or a year.

According to an embodiment, in operation 1240, the electronic device may identify a measurement history of previously measured biometric data. For example, the electronic device may identify a measurement history of biometric data measured before the missing day and/or a measurement history of biometric data measured after the missing day. For example, the electronic device may identify a measurement history of biometric data measured during another period corresponding to the missing period.

According to an embodiment, in operation 1250, the electronic device may receive text data associated with behavior of the user during the period from the user. According to an embodiment, the electronic device may determine whether additional data is required to generate missing biometric data. For example, when the additional data is not required to generate the missing biometric data, the electronic device may omit operation 1250. According to an embodiment, the electronic device may obtain the text data from the user through an input via a user interface, an input via a physical key, and/or a voice input via a microphone.

According to an embodiment, in operation 1260, the electronic device may generate missing biometric data based at least in part on the measurement history of the biometric data using a generative artificial intelligence model, for example, the AI model 213 of FIG. 2, the AI model 330 of FIG. 3, the AI model 430 of FIG. 4, or the AI model 930 of FIG. 9. According to an embodiment, when the text data is received in operation 1250, the electronic device may generate the missing biometric data based on the measurement history of the biometric data and/or the text data.

According to various embodiments, an order of the operations of FIG. 12 may be changed, at least some of the operations may be omitted, and/or at least one operation, for example, at least one of the operations of FIGS. 10, 11, 13, and 14, may be added.

According to an embodiment, biometric data missing during a specified cycle may be identified, and missing biometric data may be generated and used using the generative artificial intelligence model.

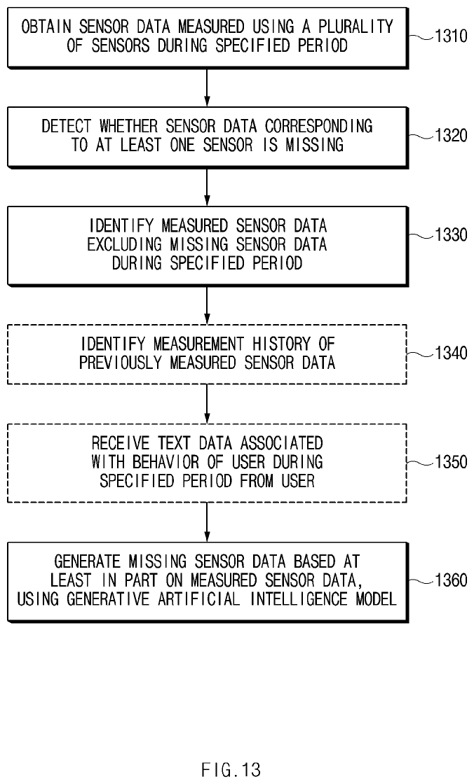

FIG. 13 is a flowchart of a method for managing data of an electronic device according to an embodiment.

In an embodiment described below, respective operations may be sequentially performed, but are not necessarily limited to sequential performance. For example, an order of the respective operations may be changed, and at least two operations may be performed in parallel.

According to an embodiment, operations 1310 to 1360 may be understood as being performed by a processor, for example, the processor 150 of FIG. 1, of an electronic device, for example, the electronic device 100 of FIG. 1.

According to an embodiment, in operation 1310, an electronic device, for example, the electronic device 100 of FIG. 1, the electronic device 210 of FIG. 2, or the electronic device 1501 of FIG. 15, may obtain sensor data measured using a plurality of sensors during a specified period, which may be referred to as a “session” in the disclosure. For example, the specified period may be specified as a different period based on a user input, a state of the electronic device, for example, a log, an application execution state, and/or a state of the user, for example, an exercise state, a sleep state, or an activity state, as determined by the electronic device.

According to an embodiment, in operation 1320, the electronic device may determine whether sensor data corresponding to at least one sensor is missing. For example, the electronic device may determine whether data of at least one sensor among the plurality of sensors used to generate information, for example, exercise information or health information, associated with the user during the specified period is missing.

According to an embodiment, in operation 1330, the electronic device may identify measured sensor data excluding missing sensor data during the specified period. For example, the electronic device may identify sensor data measured by other sensors other than the sensor corresponding to the missing sensor data among the plurality of sensors.

According to an embodiment, in operation 1340, the electronic device may identify a measurement history of previously measured sensor data. For example, the electronic device may identify a measurement history of sensor data measured during a previous period corresponding to the specified period or session. According to an embodiment, the electronic device may determine whether additional data is required to generate missing sensor data. For example, when the additional data is not required to generate the missing sensor data, the electronic device may omit operation 1340.

According to an embodiment, in operation 1350, the electronic device may receive text data associated with behavior of the user during the specified period from the user. According to an embodiment, the electronic device may determine whether additional data is required to generate missing sensor data. For example, when the additional data is not required to generate the missing sensor data, the electronic device may omit operation 1350. According to an embodiment, the electronic device may obtain the text data from the user through an input via a user interface, an input via a physical key, and/or a voice input via a microphone.

According to an embodiment, in operation 1360, the electronic device may generate missing sensor data based at least in part on the measured sensor data using a generative artificial intelligence model, for example, the AI model 213 of FIG. 2, the AI model 330 of FIG. 3, the AI model 430 of FIG. 4, or the AI model 930 of FIG. 9. According to an embodiment, the electronic device may generate the missing sensor data based on sensor data obtained through another sensor other than the sensor corresponding to the missing sensor data in the same session, sensor data obtained in a different session, and/or text data obtained from the user.

According to various embodiments, an order of the operations of FIG. 13 may be changed, at least some of the operations may be omitted, and/or at least one operation, for example, at least one of the operations of FIGS. 10 to 12 and 14, may be added.

According to an embodiment, sensor data missing during a specified cycle may be identified, and missing sensor data may be generated and used using the generative artificial intelligence model.

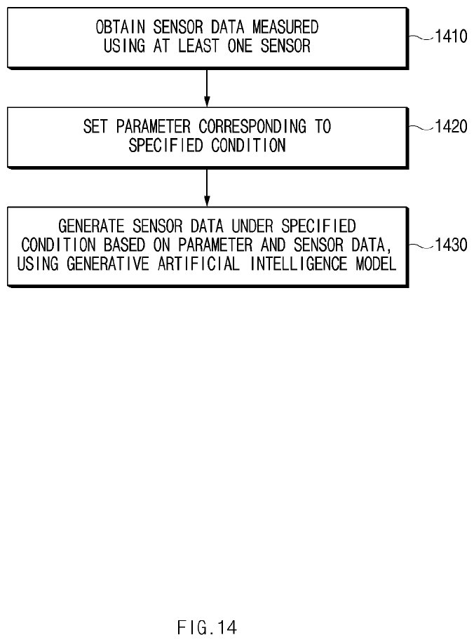

FIG. 14 is a flowchart of a method for managing data of an electronic device according to an embodiment.

In an embodiment described below, respective operations may be sequentially performed, but are not necessarily limited to sequential performance. For example, an order of the respective operations may be changed, and at least two operations may be performed in parallel.

According to an embodiment, operations 1410 to 1430 may be understood as being performed by a processor, for example, the processor 150 of FIG. 1, of an electronic device, for example, the electronic device 100 of FIG. 1.

According to an embodiment, in operation 1410, an electronic device, for example, the electronic device 100 of FIG. 1, the electronic device 210 of FIG. 2, or the electronic device 1501 of FIG. 15, may obtain sensor data measured using at least one sensor. For example, the electronic device may obtain sensor data using a sensor included in the electronic device and/or may receive sensor data from an external electronic device.

According to an embodiment, in operation 1420, the electronic device may set a parameter corresponding to a specified condition. For example, the electronic device may set a parameter corresponding to a virtual health state. For example, because electrocardiogram (ECG) data may be measured when irregular heartbeats occur in patients with atrial fibrillation in order to collect ECG data associated with atrial fibrillation, desired ECG data may be obtained. In this case, because the number of patients with atrial fibrillation may be relatively small and a time at which irregular heartbeats occur may be limited even in such patients, it may be difficult to collect the desired ECG data. The electronic device may set a parameter corresponding to a specified condition, for example, an atrial fibrillation state, based on information associated with a feature of the atrial fibrillation. For example, the electronic device may set a parameter corresponding to a virtual exercise state, for example, anaerobic exercise, aerobic exercise, strength exercise, exercise with a relatively large amount of movement, or exercise with a relatively small amount of movement. For example, sensor data of a sensor, for example, a PPG sensor, may vary depending on the exercise state.

According to an embodiment, in operation 1430, the electronic device may generate sensor data under the specified condition based on the parameter and the sensor data using a generative artificial intelligence model, for example, the AI model 213 of FIG. 2, the AI model 330 of FIG. 3, the AI model 430 of FIG. 4, or the AI model 930 of FIG. 9. For example, the electronic device may apply the set parameter, for example, a parameter corresponding to the atrial fibrillation state, to measured sensor data, for example, ECG data in a normal state, to generate sensor data under the specified condition, for example, ECG data in the atrial fibrillation state, using the generative artificial intelligence model. For example, the electronic device may generate sensor data corresponding to a virtual exercise state, for example, anaerobic exercise, aerobic exercise, strength exercise, exercise with a relatively large amount of movement, or exercise with a relatively small amount of movement, using the generative artificial intelligence model.

According to various embodiments, an order of the operations of FIG. 14 may be changed, at least some of the operations may be omitted, and/or at least one operation, for example, at least one of the operations of FIGS. 10 to 13, may be added.

According to an embodiment, the electronic device may generate sensor data corresponding to a virtual condition in various ways, thereby generating sensor data usable to develop, analyze, and/or evaluate an algorithm even in a limited environment.

According to an embodiment, a method for managing data of an electronic device may include identifying biometric data periodically measured using at least one sensor.

According to an embodiment, the method may include determining whether the biometric data is missing. For example, the method may include determining whether the biometric data is missing based on the identifying of the biometric data of the user.

According to an embodiment, the method may include identifying information related to a period during which the biometric data is missing.

According to an embodiment, the method may include identifying a measurement history of previously measured biometric data.

According to an embodiment, the method may include generating, using a generative artificial intelligence model, missing biometric data based at least in part on the measurement history.

According to an embodiment, the method may include receiving text data associated with behavior of a user during the period from the user.

According to an embodiment, generating the missing biometric data may include generating the missing biometric data based on the text data and the measurement history.

According to an embodiment, the at least one sensor may include a plurality of sensors.

According to an embodiment, the method may include obtaining sensor data measured using the plurality of sensors during a specified period.

According to an embodiment, the method may include determining whether sensor data corresponding to at least one sensor among the plurality of sensors is missing.

According to an embodiment, the method may include identifying measured sensor data excluding missing sensor data during the specified period.

According to an embodiment, the method may include generating, using the generative artificial intelligence model, the missing sensor data based at least in part on the measured sensor data.

According to an embodiment, the method may include identifying a measurement history of sensor data measured in a previous measurement period corresponding to the specified period.

According to an embodiment, generating the missing sensor data may include generating the missing sensor data based at least in part on the measurement history.

According to an embodiment, the method may include receiving text data associated with behavior of a user during the specified period from the user.

According to an embodiment, generating the missing sensor data may include generating the missing sensor data based at least in part on the text data.

According to an embodiment, the method may include obtaining at least some of biometric data or sensor data obtained through a sensor of an external electronic device from the external electronic device.

According to an embodiment, the method may include determining whether data is missing for the obtained biometric data or sensor data.

According to an embodiment, the method may include generating, using the generative artificial intelligence model, missing data for the obtained biometric data or sensor data.

According to an embodiment, the method may include comparing, for each specified cycle, a time during which the biometric data is missing with a specified threshold time based on at least one of an amount of time during which the biometric data is measured or an operation state of the electronic device.

According to an embodiment, the method may include determining that the biometric data is missing based on the time exceeds the specified threshold time.

According to an embodiment, the method may include outputting, through a display, a user interface including the measured biometric data and information indicating whether the biometric data is missing.

According to an embodiment, the method may include receiving, through the user interface, a user input confirming whether to use the generated missing biometric data based on the missing biometric data is generated.

According to an embodiment, the method may include storing the generated missing biometric data in a memory based on the user input confirming whether to use the generated missing biometric data.

According to an embodiment, the method may include receiving a user input for modifying the generated missing biometric data or the generated missing sensor data.

According to an embodiment, the method may include storing the modified missing biometric data or modified missing sensor data in a memory based on the user input for modifying the biometric data or the sensor data.

According to an embodiment, the method may include obtaining sensor data measured using the at least one sensor.

According to an embodiment, the method may include setting a parameter corresponding to a specified condition.

According to an embodiment, the method may include generating, using the generative artificial intelligence model, sensor data under the condition based on the parameter and the obtained sensor data.

According to embodiments of the disclosure, biometric data and/or sensor data missing during a specified cycle or a specified period may be identified, and missing biometric data and/or missing sensor data may be generated and used using the generative artificial intelligence model. According to embodiments of the disclosure, sensor data corresponding to a virtual condition may be generated in various ways, thereby generating sensor data usable to develop, analyze, and/or evaluate an algorithm even in a limited environment.

FIG. 15 is a block diagram illustrating an electronic device 1501 in a network environment 1500 according to various embodiments. Referring to FIG. 15, the electronic device 1501 in the network environment 1500 may communicate with an electronic device 1502 via a first network 1598, for example, a short-range wireless communication network, or with at least one of an electronic device 1504 or a server 1508 via a second network 1599, for example, a long-range wireless communication network. According to an embodiment, the electronic device 1501 may communicate with the electronic device 1504 via the server 1508. According to an embodiment, the electronic device 1501 may include a processor 1520, a memory 1530, an input module 1550, a sound output module 1555, a display module 1560, an audio module 1570, a sensor module 1576, an interface 1577, a connecting terminal 1578, a haptic module 1579, a camera module 1580, a power management module 1588, a battery 1589, a communication module 1590, a subscriber identification module (SIM) 1596, and/or an antenna module 1597. In some embodiments, at least one of the components, for example, the connecting terminal 1578, may be omitted from the electronic device 1501, or one or more other components may be added to the electronic device 1501. In some embodiments, some of the components, for example, the sensor module 1576, the camera module 1580, or the antenna module 1597, may be implemented as a single component, for example, the display module 1560.

The processor 1520 may execute, for example, software, such as a program 1540, to control at least one other component, for example, a hardware or software component, of the electronic device 1501 coupled with the processor 1520 and may perform various data processing or computation. According to one embodiment, as at least part of the data processing or computation, the processor 1520 may store, in volatile memory 1532, a command or data received from another component, for example, the sensor module 1576 or the communication module 1590, process the command or the data stored in the volatile memory 1532, and store resulting data in non-volatile memory 1534. According to an embodiment, the processor 1520 may include a main processor 1521, for example, a central processing unit (CPU) or an application processor (AP), and/or an auxiliary processor 1523, for example, a graphics processing unit (GPU), a neural processing unit (NPU), an image signal processor (ISP), a sensor hub processor, or a communication processor (CP), that is operable independently from, or in conjunction with, the main processor 1521. For example, when the electronic device 1501 includes the main processor 1521 and the auxiliary processor 1523, the auxiliary processor 1523 may consume less power than the main processor 1521 or may be dedicated to a specified function. The auxiliary processor 1523 may be implemented separately from, or as part of, the main processor 1521.

The auxiliary processor 1523 may control at least some functions or states related to at least one component, for example, the display module 1560, the sensor module 1576, or the communication module 1590, among the components of the electronic device 1501 instead of the main processor 1521 while the main processor 1521 is in an inactive state, for example, a sleep state, or together with the main processor 1521 while the main processor 1521 is in an active state, for example, while executing an application. According to an embodiment, the auxiliary processor 1523, for example, an image signal processor or a communication processor, may be implemented as part of another component, for example, the camera module 1580 or the communication module 1590, functionally related to the auxiliary processor 1523. According to an embodiment, the auxiliary processor 1523, for example, the neural processing unit, may include a hardware structure specified for artificial intelligence model processing. An artificial intelligence model may be generated by machine learning. Such learning may be performed, for example, by the electronic device 1501 on which the artificial intelligence is performed or via a separate server, for example, the server 1508. Learning algorithms may include, but are not limited to, supervised learning, unsupervised learning, semi-supervised learning, and/or reinforcement learning. The artificial intelligence model may include a plurality of artificial neural-network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), a deep Q-network, or a combination of two or more thereof, but is not limited thereto. The artificial intelligence model may additionally or alternatively include a software structure other than the hardware structure.

The memory 1530 may store various data used by at least one component, for example, the processor 1520 or the sensor module 1576, of the electronic device 1501. The various data may include, for example, software, such as the program 1540, and input data or output data for a command related thereto. The memory 1530 may include the volatile memory 1532 and/or the non-volatile memory 1534.

The program 1540 may be stored in the memory 1530 as software and may include, for example, an operating system (OS) 1542, middleware 1544, and/or an application 1546.

The input module 1550 may receive a command or data to be used by another component, for example, the processor 1520, of the electronic device 1501 from the outside, for example, from a user, of the electronic device 1501. The input module 1550 may include, for example, a microphone, a mouse, a keyboard, a key, for example, a button, or a digital pen, for example, a stylus pen.

The sound output module 1555 may output sound signals to the outside of the electronic device 1501. The sound output module 1555 may include, for example, a speaker or a receiver. The speaker may be used for general purposes, such as playing multimedia or playing recorded content. The receiver may be used for receiving incoming calls. According to an embodiment, the receiver may be implemented separately from, or as part of, the speaker.

The display module 1560 may visually provide information to the outside, for example, to a user, of the electronic device 1501. The display module 1560 may include, for example, a display, a hologram device, or a projector, and control circuitry to control a corresponding one of the display, hologram device, and projector. According to an embodiment, the display module 1560 may include a touch sensor configured to detect a touch or a pressure sensor configured to measure an intensity of force incurred by the touch.

The audio module 1570 may convert sound into an electrical signal and vice versa. According to an embodiment, the audio module 1570 may obtain sound via the input module 1550 or output sound via the sound output module 1555 or a headphone of an external electronic device, for example, the electronic device 1502, directly coupled with the electronic device 1501, for example, in a wired manner, or wirelessly coupled with the electronic device 1501.

The sensor module 1576 may detect an operational state, for example, power or temperature, of the electronic device 1501 or an environmental state, for example, a state of a user, external to the electronic device 1501 and may generate an electrical signal or data value corresponding to the detected state. According to an embodiment, the sensor module 1576 may include, for example, a gesture sensor, a gyroscope sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1577 may support one or more specified protocols to be used for coupling the electronic device 1501 with an external electronic device, for example, the electronic device 1502, directly, for example, in a wired manner, or wirelessly. According to an embodiment, the interface 1577 may include, for example, a high-definition multimedia interface (HDMI), a universal serial bus (USB) interface, a secure digital (SD) card interface, or an audio interface.

The connecting terminal 1578 may include a connector via which the electronic device 1501 may be physically connected with an external electronic device, for example, the electronic device 1502. According to an embodiment, the connecting terminal 1578 may include, for example, an HDMI connector, a USB connector, an SD card connector, or an audio connector, for example, a headphone connector.

The haptic module 1579 may convert an electrical signal into a mechanical stimulus, for example, vibration or movement, or an electrical stimulus that may be recognized by a user through tactile sensation or kinesthetic sensation. According to an embodiment, the haptic module 1579 may include, for example, a motor, a piezoelectric element, or an electric stimulator.

The camera module 1580 may capture a still image or moving images. According to an embodiment, the camera module 1580 may include one or more lenses, image sensors, image signal processors, and/or flashes.

The power management module 1588 may manage power supplied to the electronic device 1501. According to one embodiment, the power management module 1588 may be implemented as at least part of, for example, a power management integrated circuit (PMIC).

The battery 1589 may supply power to at least one component of the electronic device 1501. According to an embodiment, the battery 1589 may include, for example, a primary cell that is not rechargeable, a secondary cell that is rechargeable, or a fuel cell.

The communication module 1590 may support establishment of a direct, for example, wired, communication channel or a wireless communication channel between the electronic device 1501 and an external electronic device, for example, the electronic device 1502, the electronic device 1504, or the server 1508, and may support communication via the established communication channel. The communication module 1590 may include one or more communication processors operable independently from the processor 1520, for example, the application processor, and may support direct, for example, wired, communication or wireless communication. According to an embodiment, the communication module 1590 may include a wireless communication module 1592, for example, a cellular communication module, a short-range wireless communication module, or a global navigation satellite system (GNSS) communication module, and/or a wired communication module 1594, for example, a local area network (LAN) communication module or a power line communication (PLC) module. A corresponding one of these communication modules may communicate with the external electronic device via the first network 1598, for example, a short-range communication network such as Bluetooth™, Wi-Fi Direct, or infrared data association (IrDA), or the second network 1599, for example, a long-range communication network such as a legacy cellular network, a 5G network, a next-generation communication network, the Internet, or a computer network such as a LAN or a wide area network (WAN). These various communication modules may be implemented as a single component, for example, a single chip, or as multiple components, for example, multiple chips, separate from one another. The wireless communication module 1592 may identify and authenticate the electronic device 1501 in a communication network, such as the first network 1598 or the second network 1599, using subscriber information, for example, an international mobile subscriber identity (IMSI), stored in the subscriber identification module 1596.

The wireless communication module 1592 may support a 5G network following a 4G network and next-generation communication technology, for example, new radio (NR) access technology. The NR access technology may support enhanced mobile broadband (eMBB), massive machine-type communications (mMTC), or ultra-reliable and low-latency communications (URLLC). The wireless communication module 1592 may support a high-frequency band, for example, a mmWave band, to achieve a high data transmission rate. The wireless communication module 1592 may support various technologies for securing performance in a high-frequency band, such as beamforming, massive multiple-input and multiple-output (massive MIMO), full-dimensional MIMO (FD-MIMO), array antenna technology, analog beamforming, or large-scale antenna technology. The wireless communication module 1592 may support various requirements specified for the electronic device 1501, an external electronic device, for example, the electronic device 1504, or a network system, for example, the second network 1599. According to an embodiment, the wireless communication module 1592 may support a peak data rate, for example, 20 Gbps or more, for implementing eMBB, loss coverage, for example, 164 dB or less, for implementing mMTC, or U-plane latency, for example, 0.5 ms or less for each of downlink and uplink, or a round trip of 15 ms or less, for implementing URLLC.

The antenna module 1597 may transmit or receive a signal or power to or from the outside, for example, an external electronic device, of the electronic device 1501. According to an embodiment, the antenna module 1597 may include an antenna including a radiating element composed of a conductive material or a conductive pattern formed in or on a substrate, for example, a printed circuit board (PCB). According to an embodiment, the antenna module 1597 may include a plurality of antennas, for example, array antennas. In such a case, at least one antenna appropriate for a communication scheme used in the communication network, such as the first network 1598 or the second network 1599, may be selected, for example, by the communication module 1590, for example, the wireless communication module 1592, from among the plurality of antennas. The signal or power may then be transmitted or received between the communication module 1590 and the external electronic device via the selected at least one antenna. According to an embodiment, another component, for example, a radio frequency integrated circuit (RFIC), in addition to the radiating element, may be formed as part of the antenna module 1597.

According to various embodiments, the antenna module 1597 may form a mmWave antenna module. According to an embodiment, the mmWave antenna module may include a printed circuit board, an RFIC disposed on a first surface, for example, a bottom surface, of the printed circuit board or adjacent to the first surface and capable of supporting a designated high-frequency band, for example, a mmWave band, and a plurality of antennas, for example, array antennas, disposed on a second surface, for example, a top surface or a side surface, of the printed circuit board or adjacent to the second surface and capable of transmitting or receiving signals of the designated high-frequency band.

At least some of the above-described components may be mutually coupled and may communicate signals, for example, commands or data, therebetween via an inter-peripheral communication scheme, for example, a bus, general-purpose input and output (GPIO), serial peripheral interface (SPI), or mobile industry processor interface (MIPI).

According to an embodiment, commands or data may be transmitted or received between the electronic device 1501 and the external electronic device 1504 via the server 1508 coupled with the second network 1599. Each of the electronic devices 1502 or 1504 may be a device of the same type as, or a different type from, the electronic device 1501. According to an embodiment, all or some operations to be executed by the electronic device 1501 may be executed by one or more of the external electronic devices 1502, 1504, or 1508. For example, if the electronic device 1501 is to perform a function or service automatically, or in response to a request from a user or another device, the electronic device 1501, instead of, or in addition to, executing the function or service, may request one or more external electronic devices to perform at least part of the function or service. The one or more external electronic devices receiving the request may perform the requested part of the function or service, or an additional function or service related to the request, and may transfer an outcome of the performance to the electronic device 1501. The electronic device 1501 may provide the outcome, with or without further processing of the outcome, as at least part of a reply to the request. To that end, cloud computing, distributed computing, mobile edge computing (MEC), or client-server computing technology may be used, for example. The electronic device 1501 may provide ultra-low-latency services using, for example, distributed computing or mobile edge computing. In another embodiment, the external electronic device 1504 may include an Internet-of-Things (IOT) device. The server 1508 may be an intelligent server using machine learning and/or a neural network. According to an embodiment, the external electronic device 1504 or the server 1508 may be included in the second network 1599. The electronic device 1501 may be applied to intelligent services, for example, smart home, smart city, smart car, or healthcare services, based on 5G communication technology or IoT-related technology.

According to various embodiments, the electronic device may be one of various types of electronic devices. For example, the electronic device may include a portable communication device, for example, a smartphone, a computer device, a portable multimedia device, a portable medical device, a camera, a wearable device, or a home appliance. According to an embodiment of the disclosure, the electronic device is not limited to the examples described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the item unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C” may include any one of, or all possible combinations of, the items enumerated together in the corresponding phrase. As used herein, such terms as “1st” and “2nd,” or “first” and “second,” may be used merely to distinguish one component from another and do not limit the components in any other aspect, for example, importance or order. It is to be understood that if an element, for example, a first element, is referred to, with or without the term “operatively” or “communicatively,” as “coupled with,” “coupled to,” “connected with,” or “connected to” another element, for example, a second element, the first element may be coupled to the second element directly, for example, in a wired manner, wirelessly, or via a third element.

As used in connection with various embodiments of the disclosure, the term “module” may include a unit implemented in hardware, software, or firmware and may be interchangeably used with other terms, for example, “logic,” “logic block,” “part,” or “circuitry.” A module may be a single integral component or a minimum unit or part thereof configured to perform one or more functions. For example, according to an embodiment, a module may be implemented in the form of an application-specific integrated circuit (ASIC).

Various embodiments set forth herein may be implemented as software, for example, the program 1540, including one or more instructions stored in a storage medium, for example, internal memory 1536 or external memory 1538, that is readable by a machine, for example, the electronic device 1501. For example, a processor, for example, the processor 1520, of the machine, for example, the electronic device 1501, may invoke at least one of the one or more instructions stored in the storage medium and may execute the invoked instruction, with or without using one or more other components under control of the processor. This allows the machine to be operated to perform at least one function according to the at least one invoked instruction. The one or more instructions may include code generated by a compiler or code executable by an interpreter. The machine-readable storage medium may be provided in the form of a non-transitory storage medium. In this regard, the term “non-transitory” means that the storage medium is a tangible device and does not include a signal, for example, an electromagnetic wave, but the term does not differentiate between where data is semi-permanently stored in the storage medium and where the data is temporarily stored in the storage medium.

According to an embodiment, a method according to various embodiments of the disclosure may be included in and provided as a computer program product. The computer program product may be traded as a product between a seller and a buyer. The computer program product may be distributed in the form of a machine-readable storage medium, for example, a compact disc read-only memory (CD-ROM), or may be distributed online, for example, downloaded or uploaded, via an application store, for example, Play Store™, or directly between two user devices, for example, smartphones. When distributed online, at least part of the computer program product may be temporarily generated or at least temporarily stored in a machine-readable storage medium, such as a memory of a manufacturer's server, a server of the application store, or a relay server.

According to various embodiments, each component, for example, a module or a program, of the above-described components may include a single entity or multiple entities, and some of the multiple entities may be separately disposed in different components. According to various embodiments, one or more of the above-described components may be omitted, or one or more other components may be added. Alternatively or additionally, a plurality of components, for example, modules or programs, may be integrated into a single component. In such a case, according to various embodiments, the integrated component may still perform one or more functions of each of the plurality of components in the same manner as or a similar manner to that in which they are performed by a corresponding one of the plurality of components before the integration. According to various embodiments, operations performed by the module, the program, or another component may be carried out sequentially, in parallel, repeatedly, or heuristically, one or more of the operations may be executed in a different order or omitted, or one or more other operations may be added.

1. An electronic device, comprising:
communication circuitry;
at least one sensor;
memory configured to store a generative artificial intelligence model; and
a processor, wherein the memory stores instructions that, when executed by the processor, cause the electronic device to:
identify biometric data of a user periodically measured using the at least one sensor;
determine whether the biometric data is missing based on the identifying of the biometric data of the user;
identify information related to a period during which the biometric data was missing;
identify a measurement history of previously measured biometric data; and
generate, using the generative artificial intelligence model, missing biometric data based at least in part on the measurement history.

2. The electronic device of claim 1, wherein the instructions, when executed by the processor, cause the electronic device to:
receive text data associated with behavior of the user during the period; and
generate the missing biometric data based on the text data and the measurement history.

3. The electronic device of claim 1, wherein the at least one sensor comprises a plurality of sensors, and wherein the instructions, when executed by the processor, cause the electronic device to:
obtain sensor data measured using the plurality of sensors during a specified period;
determine whether sensor data corresponding to at least one sensor among the plurality of sensors is missing;
identify measured sensor data excluding missing sensor data during the specified period; and
generate, using the generative artificial intelligence model, missing sensor data based at least in part on the measured sensor data.

4. The electronic device of claim 3, wherein the instructions, when executed by the processor, cause the electronic device to:
identify a measurement history of sensor data measured in a previous measurement period corresponding to the specified period; and
generate the missing sensor data based at least in part on the measurement history of the sensor data.

5. The electronic device of claim 4, wherein the instructions, when executed by the processor, cause the electronic device to:
receive text data associated with behavior of a user during the specified period; and
generate the missing sensor data based at least in part on the measurement history of the sensor data and text data.

6. The electronic device of claim 3, wherein the instructions, when executed by the processor, cause the electronic device to:
obtain at least some biometric data or sensor data obtained through at least one sensor of an external electronic device from the external electronic device;
determine whether data is missing for the obtained biometric data or sensor data; and
generate, using the generative artificial intelligence model, missing data for the obtained biometric data or sensor data.

7. The electronic device of claim 1, wherein the instructions, when executed by the processor, cause the electronic device to:
compare, for each specified cycle, a time during which the biometric data is missing with a specified threshold time based on at least one of an amount of time during which the biometric data is measured or an operation state of the electronic device; and
determine that the biometric data is missing based on the time exceeds the specified threshold time.

8. The electronic device of claim 1, wherein the instructions, when executed by the processor, cause the electronic device to:
output, through a display of the electronic device, a user interface including the measured biometric data and information indicating whether the biometric data is missing;
receive, through the user interface, a user input confirming whether to use the generated missing biometric data based on the missing biometric data is generated; and store the generated missing biometric data in the memory based on the user input.

9. The electronic device of claim 3, wherein the instructions, when executed by the processor, cause the electronic device to:
receive a user input for modifying the generated missing sensor data; and
store the modified missing sensor data in the memory based on the user input.

10. The electronic device of claim 1, wherein the instructions, when executed by the processor, cause the electronic device to:
obtain sensor data measured using the at least one sensor;
set a parameter corresponding to a specified condition; and
generate, using the generative artificial intelligence model, sensor data under the specified condition based on the parameter and the obtained sensor data.

11. A method for managing data of an electronic device, the method comprising:
identifying biometric data periodically measured using at least one sensor;
determining whether the biometric data is missing based on the identifying of the biometric data of the user;
identifying information related to a period during which the biometric data was missing;
identifying a measurement history of previously measured biometric data; and
generating, using a generative artificial intelligence model, missing biometric data based at least in part on the measurement history.

12. The method of claim 11, further comprising receiving text data associated with behavior of a user during the period, wherein generating the missing biometric data comprises generating the missing biometric data based on the text data and the measurement history.

13. The method of claim 11, wherein the at least one sensor comprises a plurality of sensors, the method further comprising:
obtaining sensor data measured using the plurality of sensors during a specified period;
determining whether sensor data corresponding to at least one sensor among the plurality of sensors is missing;
identifying measured sensor data excluding missing sensor data during the specified period; and
generating, using the generative artificial intelligence model, missing sensor data based at least in part on the measured sensor data.

14. The method of claim 13, further comprising identifying a measurement history of sensor data measured in a previous measurement period corresponding to the specified period, wherein generating the missing sensor data comprises generating the missing sensor data based at least in part on the measurement history of the sensor data.

15. The method of claim 13, further comprising receiving text data associated with behavior of a user during the specified period, wherein generating the missing sensor data comprises generating the missing sensor data based at least in part on the text data.

16. The method of claim 13, further comprising:
obtaining at least some biometric data or sensor data obtained through at least one sensor of an external electronic device from the external electronic device;
determining whether data is missing for the obtained biometric data or sensor data; and
generating, using the generative artificial intelligence model, missing data for the obtained biometric data or sensor data.

17. The method of claim 11, further comprising:
comparing, for each specified cycle, a time during which the biometric data is missing with a specified threshold time based on at least one of an amount of time during which the biometric data is measured or an operation state of the electronic device; and
determining that the biometric data is missing based on the time exceeds the specified threshold time.

18. The method of claim 11, further comprising:
outputting, through a display of the electronic device, a user interface including the measured biometric data and information indicating whether the biometric data is missing;
receiving, through the user interface, a user input confirming whether to use the generated missing biometric data based on the missing biometric data is generated; and
storing the generated missing biometric data in a memory of the electronic device based on the user input.

19. The method of claim 13, further comprising:
receiving a user input for modifying the generated missing sensor data; and
storing the modified missing sensor data in a memory of the electronic device based on the user input.

20. The method of claim 11, further comprising:
obtaining sensor data measured using the at least one sensor;
setting a parameter corresponding to a specified condition; and
generating, using the generative artificial intelligence model, sensor data under the specified condition based on the parameter and the obtained sensor data.
